(19) United States
(12) Patent Application Publication
(10) Pub. No.: US 2026/0076906 A1
(43) Pub. Date: Mar. 19, 2026

(54) Nanoparticles for Enhancing Mitochondrial Networks

(71) Applicant: Northeastern University, Boston, MA (US)

(72) Inventors: Mansoor AMIJI, Attleboro, MA (US); Lara Milane, Boston, MA (US)

(21) Appl. No.: 19/172,228

(22) Filed: Apr. 7, 2025

Related U.S. Application Data
(60) Provisional application No. 63/575,610, filed on Apr. 5, 2024.

(51) Int. Cl. A61K 9/1272; A61K 9/51; A61K 38/46; A61P 43/00

(52) U.S. Cl. CPC A61K 9/1272; A61K 9/5123; A61K 38/46; A61P 43/00; C12Y 306/05

(57) ABSTRACT

The present technology provides compositions and methods for enhancing mitochondrial networks in cells, particularly mammalian cells, and preferably human cells. The compositions and methods incorporate nanoparticles each including a mitochondrial network enhancing agent. When the nanoparticles are taken by the cell, the mitochondrial network enhancing agent binds to the mitofusin (MFN) proteins 1 and 2 thereby promoting fusion of mitochondria of the cell with one another and/or with endoplasmic reticulum of the cell. The present technology's compositions and methods find use in in the treatment of neurological, mitochondrial dysfunction, metabolic, and/or accelerated aging diseases or disorders including, for non-limiting examples, a progeroid syndrome, an MFN related disorder, Marie-Charcot Tooth disease, or any combination thereof.

### CROSS-REFERENCE TO RELATED APPLICATIONS

This application claims priority to U.S. Provisional Application No. 63/575,610, filed 5 Apr. 2024, which is incorporated by reference herein in its entirety.

### BACKGROUND

Mitochondrial dysfunction is central to neurodegenerative disease and the natural aging process as mitochondria are the mediators of cell death (intrinsic apoptosis), energy production, and reactive oxygen species (ROS) generation—all of which are altered during aging and neurodegenerative disease.

Alzheimer's Disease (AD) and related diseases are hallmarked by protein accumulation (such as β-amyloid and Tau), oxidative stress, calcium imbalance, and premature neuronal cell death. The endoplasmic reticulum stores calcium and mediates protein synthesis and folding while mitochondria produce energy adenosine triphosphate (ATP) (which can yield damaging reactive oxygen species) and mediate cell death through the intrinsic apoptotic pathway. Mitochondria are in a fluctuating state of fusion and fission with each other and with the endoplasmic reticulum. Mitochondrial network fusion has been shown to occur in response to high energy demand, mitochondrial fission is required for cell death, and mitochondrial fusion to the endoplasmic reticulum results in coupling cellular energy (mitochondrial ATP) to the energetic demanding process of protein synthesis (the endoplasmic reticulum function).

Mitochondrial fusion is mediated by the mitofusin (MFN) proteins 1 and 2, with a predominance of mitofusin 2 (MFN2) mediation^{2,3}. Mitochondrial fusion increases oxidative phosphorylation (OXPHOS) capacity³⁻⁶. Mitofusin 2 (MFN2) also mediates mitochondrial fusion with the endoplasmic reticulum. The endoplasmic reticulum is responsible for protein synthesis and folding; as cells are continually adapting and evolving to ensure their survival, there is a constant demand for high levels of protein synthesis. Mitochondrial fusion to the endoplasmic reticulum directly couples energy supply to the energy demanding process of protein synthesis, enabling growing and aging cells to continue to survive challenges through responsive alterations in protein expressions. A second implication of mitochondrial-endoplasmic reticulum fusion is mitochondrial rescue of the endoplasmic reticulum from the unfolded protein response (UPR)¹²⁻¹⁶. The unfolded protein response is a scripted process that can lead to cell death or survival¹⁷. Endoplasmic reticulum stress increases mitochondrial-endoplasmic reticulum fusion, Ca²⁺ exchange, and oxidative phosphorylation¹⁸. The unfolded protein response can trigger mitophagy and autophagy and lead to cellular repair while mitochondrial-endoplasmic reticulum Ca²⁺ exchange increases metabolism and survival¹⁹.

### SUMMARY

The present technology provides compositions and methods for enhancing mitochondrial networks in cells, particularly mammalian cells, and preferably human cells. The compositions and methods incorporate nanoparticles each including a mitochondrial network enhancing agent. When the nanoparticles are taken up by the cell, the mitochondrial network enhancing agent binds to the mitofusin (MFN) proteins 1 and 2 thereby promoting fusion of mitochondria of the cell with one another and/or with endoplasmic reticulum of the cell. This enhancement of the cellular mitochondrial network enhances a multifactorial cascade of cellular consequences including increased oxidative phosphorylation capacity, decreased reactive oxygen species, resistance to cell death signaling, increased cellular recycling, increased protein folding efficiency, and resolution of the unfolded protein response. The present technology's compositions and methods find use in in the treatment of neurological disease, mitochondrial dysfunction, metabolic, autoimmune and/or accelerated aging diseases or disorders including, for non-limiting examples, Alzheimer's disease, Parkinson's disease, Diabetes, a progeria, an auto-immune disease such as Lupus, a mitofusin (MFN) specific disorder such as Marie-Charcot Tooth disease, a mitochondrial genetic disease such as mitochondrial encephalomyopathy lactic acidosis and stroke-like episodes (MELAS,) or any combination thereof.

In one aspect, the present technology provides a composition for enhancing a mitochondrial network in a cell. The composition includes a plurality of nanoparticles each comprising a mitochondrial network enhancing agent; wherein the mitochondrial network enhancing agent enhances the mitochondrial network of the cell when the nanoparticles are taken up by the cell.

The technology can be further summarized in the following list of features:

1. A composition for enhancing a mitochondrial network in a cell, the composition comprising:
a plurality of nanoparticles each comprising a mitochondrial network enhancing agent;
wherein the mitochondrial network enhancing agent enhances the mitochondrial network of the cell when the nanoparticles are taken up by the cell.

2. The composition of feature 1, wherein the mitochondrial network enhancing agent binds to a mitofusin protein on mitochondria of the cell.

3. The composition of feature 2, wherein the mitofusin protein is mitofusin 1 (MFN1) or mitofusin 2 (MFN2).

4. The composition of feature 3, wherein the mitofusin protein is MFN2.

5. The composition of feature 3, wherein the mitochondrial network enhancing agent binds to MFN1 or MFN2 and thereby promotes fusion of mitochondria of the cell with one another and/or with endoplasmic reticulum of the cell.

6. The composition of feature 5, wherein the mitochondrial network enhancing agent is a peptide fragment of MFN1 or MFN2.

7. The composition of feature 6, wherein the mitochondrial network enhancing agent is a peptide fragment of MFN2 and comprises the amino acid sequence DIAEAVRLIMDSLHMAAR (SEQ ID NO:1), or a fragment or variant thereof having at least 70%, at least 80%, at least 90%, at least 95%, at least 98%, or at least 99% identity to SEQ ID NO:1.

8. The composition of feature 7, wherein the mitochondrial network enhancing agent is a variant of SEQ ID NO: 1 comprising from 1 to 10, from 1 to 5, or from 1 to 3 conservative amino acid substitutions compared to SEQ ID NO:1.

9. The composition of any of the preceding features, wherein the nanoparticles comprise lipid nanoparticles or biodegradable polymeric nanoparticles, and wherein the lipid nanoparticles or polymeric nanoparticles encapsulate the mitochondrial network enhancing agent.

10. The composition of any of the preceding features, wherein the nanoparticles comprise lipid nanoparticles, and wherein the lipid nanoparticles are liposomes.

11. The composition of feature 10, wherein the liposomes are cationic liposomes.

12. The composition of feature 11, wherein the cationic liposomes comprise 1,2-dioleoyl-3-trimethylammonium-propane (DOTAP) as the cationic lipid and further comprise one or more components selected from the group consisting of a neutral phospholipid, cholesterol, and a hydrophilic polymer such as polyethylene glycol (PEG).

13. The composition of feature 11, wherein the cationic liposomes have a zeta potential greater than about +30 mV or less than about −30 mV.

14. The composition of feature 9, wherein the nanoparticles are biodegradable polymeric nanoparticles.

15. The composition of feature 14, wherein the biodegradable polymeric nanoparticles comprise one or more biodegradable polymeric polymers selected from the group consisting of gelatin, chitosan, cellulose, cellulose derivatives, poly(ε-caprolactone) (PCL), polyethylene glycol (PEG), poly(lactic-co-glycolic acid) (PLGA), polylactic acid (PLA), polyglycolide, PEG-PLA diblock copolymer, PEG-PLGA diblock copolymer, PEG-PCL diblock copolymer, PCL-b-PEG-b-PCL co-polymer, polypropylene glycol (PPG), polyacrylic acid, polyacrylamide, poly(N-isopropylacrylamide), polyethylene oxide-polypropylene oxide-polyethylene oxide (PEO-PPO-PEO) triblock copolymer, hyaluronic acid, polyethylene oxide, polypropylene oxide, alginic acid, poly(betaaminoester) (PBAE), poly(glycolic acid) (PGA), and alginate.

16. The composition of any of the preceding features, wherein the composition further comprises one or more excipients and is a pharmaceutical composition.

17. A method of enhancing a mitochondrial network in a cell of a mammalian subject, the method comprising:
(a) providing the composition of any of the preceding features; and
(b) administering the composition to the mammalian subject, whereby the mitochondrial network is enhanced in the cell of the mammalian subject.

18. The method of feature 17, wherein said enhancing the mitochondrial network in the cell of the mammalian subject comprises enhancing fusion of mitochondria with other mitochondria and/or with endoplasmic reticulum in the cell, and/or increasing a size or density of the mitochondrial network in the cell.

19. The method of feature 17 or feature 18, wherein said enhancing the mitochondrial network in the cell of the mammalian subject results in one or more of increased energy production, reduced reactive oxygen species (ROS) production, reduced nuclear DNA damage, reduced mitochondrial DNA damage, increased cell survival, increased protein folding efficiency, reduced protein misfolding, reduced accumulation of misfolded proteins, increased autophagy, increased mitophagy, and reduced apoptosis.

20. The method of any of features 17-19, wherein said enhancing the mitochondrial network in the cell of the mammalian subject comprises reducing fission of mitochondria in cells of the mammalian subject.

21. The method of any of features 17-20, wherein the mammalian subject is a human subject.

22. The method of feature 21, wherein the human subject has a neurological disease or disorder, a disease or disorder related to mitochondrial dysfunction, a metabolic disease or disorder, an accelerated aging disorder, a progeroid syndrome, an MFN related disorder, Marie-Charcot Tooth disease, an autoimmune disease, or any combination thereof.

23. The method of feature 22, wherein the neurological disease or disorder is a neurodegenerative disease.

24. The method of feature 23, wherein the neurodegenerative disease is selected from the group consisting of Alzheimer's disease, Parkinson's disease, Huntington's disease, amyotrophic lateral sclerosis (ALS), spinal muscular atrophy, a prion disease, and combinations thereof.

25. The method of feature 22, wherein the disease or disorder related to mitochondrial dysfunction is selected from the group consisting of a disease related to decrease of mitochondrial energy capacity, Charcot-Marie-Tooth disease type 2A (CMT2A), a mental health condition showing mitochondrial dysfunction, a genetic mitochondrial disease, mitochondrial encephalomyopathy, lactic acidosis and stroke-like episodes (MELAS), a secondary mitochondrial dysfunction due to another condition or disease, and combinations thereof.

26 The method of feature 22, wherein the metabolic disease or disorder is selected from the group consisting of diabetes, obesity, metabolic syndrome, and combinations thereof.

27 The method of feature 22, wherein the cardiovascular disease is selected from the group consisting of ischemia, myocardial infarction, stroke, heart failure, cardiomyopathy, and combinations thereof.

28 The method of feature 22, wherein the progeroid syndrome is selected from the group consisting of progeria, Hutchinson-Gilford Progeria Syndrome, Werner syndrome, and combinations thereof.

29. The method of feature 22, wherein the autoimmune disease is selected from the group consisting of lupus, asthma, arthritis, inflammatory bowel disease and irritable bowel syndrome, and combinations thereof.

30. The method of any of features 17-28, wherein the step of administering comprises administering said plurality of nanoparticles across a biological barrier including the blood-brain barrier for Chaetoglobosin A nanoparticles of the mammalian subject or bypassing a blood-brain barrier of the mammalian subject.

31. A method of enhancing a mitochondrial network in a cell, the method comprising:
(a) providing the composition of any of the preceding features and a culture comprising the cell; and
(b) adding the composition to a medium of the culture, whereby nanoparticles of the composition are taken up by the cell and the mitochondrial network in the cell is enhanced.

31. The method of feature 30, further comprising administering the cell to a mammalian subject in need thereof.

32 The method of feature 31, wherein the cell is an immune cell, such as a CAR-T cell.

As used herein, the reference numbers refer to Reference List 1 unless referenced in the context of Examples 9-13, where they refer to Reference List 2, or unless otherwise specified.

As used herein, the term “about” refers to a range of within plus or minus 10%, 5%, 1%, or 0.5% of the stated value.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expression “consisting of” or “consisting essentially of”.

As used herein, the phrase “nanoparticles each encapsulating a mitochondrial network enhancing agent” is used interchangeably with the phrase “mitochondrial network enhancing nanoparticles”.

As used herein, the terms “MiNE NP”, “MINE NP”, ‘MiNE LNP”, and/or “MiNE LNP” are used interchangeably in respective singular and/or plural forms and refer to poly(ethylene glycol)-modified (PEGylated), biocompatible liposome nanoparticle(s) each with an aqueous core encapsulating a validated MFN2 peptide fragment including amino acid sequence DIAEAVRGIMDSLHMAAR (SEQ ID NO:1:) with a molecular weight of 1956.26 g/mol²¹ (according to Reference List 2).

As used herein, the terms “nanoparticle” or “nanoparticles” are used interchangeably with the terms “NP” and “NPS.

As used herein, the terms “liposome” and/or “liposomes” are used interchangeably with the terms “LP” and/or LPS”.

As used herein, the term “microbial network analysis” is used interchangeably with the terms “MiNA” and/or “MINA”.

As used herein, the term “endoplasmic reticulum” is used interchangeably with the term “ER”.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic illustration of a cell showing the mechanism of action of the composition of the invention.

FIG. 2 is a characterization of cationic liposomes loaded with mitofusin 2 (MFN2) peptide fragment including the amino acid sequence SEQ ID NO:1: as measured by Dynamic Light Scattering using a Malvern ZetaSizer as a function of intensity.

FIG. 3A shows a schematic of a predicted structure of mitofusin 2 (MFN2) peptide fragment including amino acid sequence SEQ ID NO: 1 based on the Innovagen Pep-Fold peptide structure prediction server.

FIG. 3B shows a predicted characterization of the properties an alpha-helical mitofusin 2 (MFN2) peptide fragment including the amino acid sequence SEQ ID NO: 1 based on the Innovagen peptide property calculator.

FIG. 4A is an image of mitochondria stained Mitotracker™ Green in a fluorescent channel obtained for mitochondrial network analysis.

FIG. 4B is second image and overlay on first image of FIG. 4A for mitochondrial network analysis.

FIG. 4C is the corresponding brightfield image and overlay of the first image of FIG. 4A for mitochondrial network analysis.

FIG. 4D is a converted binary image of FIG. 4A for mitochondrial network analysis.

FIG. 4E is a converted skeletonized image of FIG. 4A for mitochondrial network analysis.

FIG. 4F is a three-dimensional analysis based on FIGS. 4A-44E for mitochondrial network analysis.

FIG. 5 shows a mitochondrial network analysis of untreated mouse fibroblast (NIH3T3) cells and NIH3T3 cells treated with cationic liposomes each including mitofusin 2 (MFN2) peptide fragment including amino acid sequence (SEQ ID NO:1).

FIG. 6 shows a mitochondrial network analysis of untreated mouse fibroblast (NIH3T3) and NIH3T3 cells treated with cationic and neutral liposomes each including mitofusin 2 (MFN2) peptide fragment including amino acid sequence (SEQ ID NO:1).

FIG. 7 shows the rate of release of mitofusin 2 (MFN2) peptide fragment including amino acid sequence DIAEAVRLIMDSLHMAAR (SEQ ID NO:1 NO: 1) in solution from cationic and neutral encapsulating liposomes.

FIG. 8 shows the mean mitochondrial network size for untreated mouse fibroblast (NIH3T3) cells and NIH3T3 cells treated for 2 hours and for 6 hours with cationic liposomes loaded with mitofusin 2 (MFN2) peptide fragment including amino acid sequence (SEQ ID NO:1).

FIG. 9A shows fluorescence images of endoplasmic reticulum and mitochondria overlayed on top of a cell including a grayscale image of the cell.

FIG. 9B shows fluorescence images of endoplasmic reticulum and mitochondria overlayed on top of a cell including the endoplasmic reticulum in red.

FIG. 9C shows fluorescence images of endoplasmic reticulum and mitochondria overlayed on top of a cell including the mitochondria in green.

FIG. 9D is a cytofluorogram showing pixel co-localization of mitochondrial staining (green) and endoplasmic reticulum staining (red) using JACoP software.

FIG. 10 shows co-localization of endoplasmic reticulum and mitochondria for untreated (purple) mouse fibroblast (NIH3T3) cells and NIH3T3 cells (green) treated with cationic liposomes loaded with mitofusin 2 (MFN2) peptide fragment based on Pearson coefficient and Mander's coefficients of endoplasmic reticulum and mitochondria fluorescence channels.

FIG. 11 shows a preliminary analysis of mitochondrial networks human cortical neurons stained with Mitotracker™ Green in a fluorescent channel, at 60× magnification ex vivo after treatment with cationic liposomes loaded with mitofusin 2 (MFN2) peptide fragment including amino acid sequence (SEQ ID NO:1).

FIGS. 12A-D show microscopy images for mitochondrial network quantification of untreated mouse fibroblast NIH3T3 cells (FIG. 12A) and NIH3T3 cells treated with liposomes loaded with mitofusin 2 (MFN2) peptide fragment including amino acid sequence (SEQ ID NO:1) (MiNE NP treated cells) (FIG. 12B), NIH3T3 cells treated with non-encapsulating liposomes (Blank NP treated cells) (FIG. 12C), and NIH3T3 cells treated with MFN2 peptide in solution (Peptide Sol treated cells) (FIG. 12D).

FIG. 13A-D illustrates the parameters measured from the three-dimensional network images of FIG. 12, where FIG. 13A is a mitochondrial network analysis of individual mitochondria for the cells of FIG. 12 after two hours of treatment; FIG. 13B is a mitochondrial network analysis of the mitochondrial networks for the cells of FIG. 12 after two of treatment; FIG. 13C is a mitochondrial network analysis of the mean mitochondrial network sizes for the cells of Figure after two hours of treatment; and FIG. 13D is a mitochondrial network analysis of the mitochondrial footprints for the cells of FIG. 12 after two of treatment.

FIGS. 14A-B show microscopy results of mitochondria (Green) and endoplasmic reticulum (Red) colocalization, where FIG. 14A shows untreated NIH3T3 Cells, and FIG. 14B shows MiNE NP treated cells.

FIG. 15 shows mitochondrial-endoplasmic reticulum (ER) co-localization of untreated and MiNE NP treated cells measured through Pearson and Mander's coefficients for pixel overlap.

FIG. 16 shows Abcam's MitoTox™ Complex V Oxidative Phosphorylation (OXPHOS) Assay results with increasing concentrations of liposomes loaded with mitofusin 2 (MFN2) peptide fragment including amino acid sequence (SEQ ID NO:1) on complex v activity in purified mitochondria.

FIG. 17 shows an evaluation of the ability of the liposomes loaded with mitofusin 2 (MFN2) peptide fragment including amino acid sequence (SEQ ID NO:1) to protect against the unfolded protein response (UPR) in mouse fibroblast (NIHT3T) cells and in hippocampal neurons.

FIG. 18 is a schematic illustration showing the mechanism of action for a composition including nanoparticles including a mitochondrial network enhancing agent according to an embodiment of the present invention.

### DETAILED DESCRIPTION

The current technology relates to compositions for and methods of enhancing a mitochondrial network in a cell, wherein the composition includes nanoparticles each encapsulating a mitochondrial network enhancing agent. When the nanoparticles are taken up by a cell, the mitochondrial network enhancing agent binds to mitofusin proteins including mitofusin 1 (MFN1) and/or mitofusin 2 (MFN2) on the cell mitochondria. Preferably, the agent binds with the mitofusin 2 (MFN2) proteins. The binding of the agent to the mitofusin proteins 1 (MFN1) and/or mitofusin 2 (MFN2) promotes fusion of cellular mitochondria with one another and/or fusion of mitochondria with the endoplasmic reticulum of the cell.

The mitochondrial network enhancing nanoparticles are the first therapy for enhancing the cellular mitochondrial network including the promotion of fusion or mitochondria with one another and/or the fusion of mitochondria to the endoplasmic reticulum in a patient. The mitochondrial network enhancing agent is a peptide fragment of the proteins mitofusin 1 (MFN1) or mitofusin 2 (MFN2). The peptide fragment includes the amino acid sequence (SEQ ID NO:1) or a fragment or variant thereof having at least 70%, at least 80%, at least 90%, at least 95%, at least 98%, or at least 99% identity to SEQ ID NO:1. The variant of SEQ ID NO: 1 for use in the present technology includes from 1 to 10, preferably from 1 to 5, or more preferably from 1 to 3 conservative amino acid substitutions compared to SEQ ID NO:1.

Conservative amino acid substitutions in a peptide or polypeptide are substitutions of an amino acid with an equivalent amino acid which do not substantially alter the structure and/or functionality of the peptide or polypeptide. Equivalent amino acids have side chains with similar properties such as bulkiness, polarity (polar or non-polar), hydrophobicity (hydrophobic or hydrophilic), pH-dependent protonatable groups (acidic, neutral or basic), and organization of carbon molecules (aromatic or aliphatic).

Amino acids can be divided into the following classes, within which amino acids are considered equivalent. Substitutions made within the same class are considered conservative substitutions.
1. Amino acids having polar side chains (Asp, Glu, Lys, Arg, His, Asn, Gln, Ser, Thr, Tyr, and Cys,)
2. Amino acids having non-polar side chains (Gly, Ala, Val, Leu, Ile, Phe, Trp, Pro, and Met)
3. Amino acids having aliphatic side chains (Gly, Ala Val, Leu, Ile)
1. Amino acids having cyclic side chains (Phe, Tyr, Trp, His, Pro)
2. Amino acids having aromatic side chains (Phe, Tyr, Trp)
3. Amino acids having acidic side chains (Asp, Glu)
4. Amino acids having basic side chains (Lys, Arg, His)
5. Amino acids having amide side chains (Asn, Gln)
6 Amino acids having hydroxy side chains (Ser, Thr)
7. Amino acids having sulfur-containing side chains (Cys, Met),
8. Neutral, weakly hydrophobic amino acids (Pro, Ala, Gly, Ser, Thr)
9. Hydrophilic, acidic amino acids (Gln, Asn, Glu, Asp), and
10. Hydrophobic amino acids (Leu, Ile, Val)

The enhancement of the cellular mitochondrial network protects cells from death³⁻⁶. Mitochondrial fission is required for intrinsic apoptosis³⁻⁷. Mitochondrial fission enables pro-apoptotic Bcl-2 family members (Bax) to bind to the mitochondrial outer membrane (OMM) and begin the apoptotic cascade^{6,8-11}. During fission, foci are formed on the mitochondrial outer membrane. Bax binds to these foci and associates with mitofusin 2 (MFN2) at the scission sites⁸⁻¹¹.

The binding of the mitochondrial network enhancing agent to the proteins mitofusin 1 (MFN1) and/or mitofusin 2 (MFN2) in the cell promotes or increases the fusion of mitochondria to one and another and/or the fusion of the mitochondria to the endoplasmic reticulum thereby enhancing the cellular mitochondrial network. The enhanced cellular mitochondrial network promotes a reduction in the unfolded protein response, an increase in protein folding efficiency, and an increase in autophagic cellular repair. These changes favor cell survival, efficiency, and repair over cell death. These changes also lead to an increase in the apoptotic threshold of cells making the cells more resistant to cell death signaling, an increase in oxidative phosphorylation, and a decrease in reactive oxygen species. The higher capacity for oxidative phosphorylation is critical to improving cell survival and significantly decreasing the accumulation of DNA damage.

The mitochondrial network enhancing nanoparticles are designed for the non-limiting treatment of neurodegenerative disease, as an anti-aging therapy, and for the treatment of progeria. Neurodegenerative disease, natural aging, and accelerated aging are marked by a decrease in mitochondrial energy capacity and an increase in the accumulation of misfolded proteins. The enhancement of the mitochondrial network including the promotion of an increase of mitochondrial fusion to one another promotes an increase in mitochondrial energy capacity and health, and an increase cellular resistance to death signaling. The enhancement of the mitochondrial network including the promotion of an increase in mitochondrial to endoplasmic reticulum fusion promotes an increase in protein folding efficiency and a decrease cell stress. Compositions for and methods of enhancing the mitochondrial network are unique, unprecedented therapies for natural and accelerated aging as well as neurodegenerative disease.

Mitochondria are well known mediators of the natural aging process and neurodegenerative disease as they control cell death (apoptosis), produce most of the cell's energy (adenosine triphosphate or ATP), and are the primary contributors to deoxyribonucleic acid or DNA damage through reactive oxygen species production. Mitochondria also bind to the endoplasmic reticulum, directly supplying energy to the energy demanding process of protein synthesis. Under cell stress, during aging, and in neurodegenerative disease, there is an accumulation of misfolded proteins; mitochondrial fusion to the endoplasmic reticulum can protect against the unfolded protein response, increasing the protein folding efficiency of the cell.

The enhancement of the mitochondrial network increases the capacity for adenosine triphosphate (ATP) and protects cells against death signaling by orienting mitochondria in an anti-apoptotic conformation. Although less characterized, mitochondrial abnormalities have also been demonstrated in the accelerated aging diseases, progerias. These changes include mitochondrial network fragmentation and elevated reactive oxygen species levels¹. As mitochondria are the cellular Achilles heel of natural aging, improving mitochondrial function could offer a new treatment for aging and neurodegenerative disease.

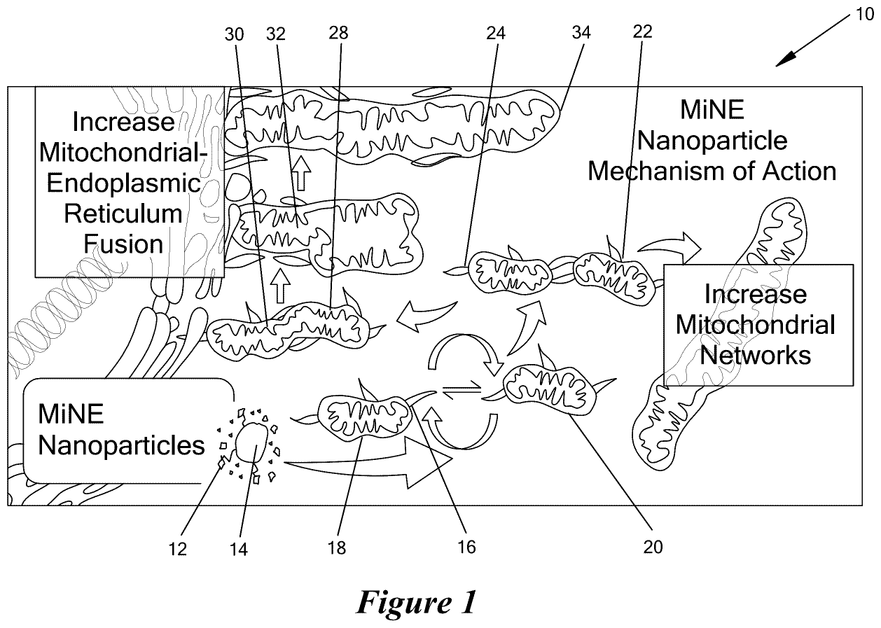

The mechanism of action for the enhancement of the mitochondrial network by the use of the present technology is illustrated schematically in FIG. 1. Nanoparticles 12 each encapsulating a mitochondrial network enhancing agent 14 are taken up by cell 10. The agent 14 released from the nanoparticle binds to a mitofusin protein 16 on mitochondria 18 which promotes fusion of mitochondria 18 with mitochondria 20 forming fused mitochondria 22. Agent 14 binds to mitofusin protein 24 of fused mitochondria 22 which promotes fusion of fused mitochondria 22 with endoplasmic reticulum 26 forming mitochondria 28 fused to the endoplasmic reticulum. Agent 14 binds to mitofusin protein 30 on mitochondria 28 fused to the endoplasmic reticulum promoting fusion with adjacent mitochondria 32 fused with endoplasmic reticulum thereby forming mitochondria 34 fused to the endoplasmic reticulum.

The functional consequence of increased fusion of mitochondria with one another and increased mitochondrial fusion with the endoplasmic reticulum include lower cellular damage and increased death resistance, increased energy production, lower reactive oxygen species production and less reactive oxygen species induced DNA damage, increased survival due to mitochondrial network conformation protecting against pro-apoptotic signaling, and increased protein folding efficiency, decreased misfolded protein accumulation, and increased autophagy (from mitochondrial fusion to the endoplasmic reticulum).

The compositions for and methods of enhancing the mitochondrial network have the potential to revolutionize the non-limiting treatment of neurodegenerative disease and the aging process by (1) improving the efficacy of protein production and folding, (2) decreasing the accumulation of misfolded proteins, (3) increasing cellular energy capacity, (4) increasing the rate of cell recycling (mitophagy and autophagy), (5) decreasing reactive oxygen species production and reactive oxygen species induced DNA damage, and (6) increasing cellular resistance to death signaling.

The nanoparticles of the present technology can be used as delivery vehicles to transport an active agent to its target cell in a variety of contexts. The nanoparticles can be introduced into the blood or lymphatic system or injected directly into the interstitial fluid of an organ or tissue, for example. In some cases, however, the nanoparticles can be administered so as to overcome or cross a biological barrier, such as the blood-brain barrier, the intestinal mucosal barrier, a basement membrane, or an epithelial cell layer. In such cases, administering the nanoparticles across the biological barrier can be achieved, for example, by penetrating the barrier with a device to perform injection across the barrier, or by implantation of a time-release composition or device that releases the nanoparticles directly across the barrier or into a target organ or tissue. Alternatively, the size or other properties of the nanoparticles can be selected so that the nanoparticles are able to cross the biological barrier. For example, small nanoparticles having a size of about 50 nm or less may be able to cross the biological barrier without further intervention.

The nanoparticles each including the mitochondrial network enhancing agent include lipid nanoparticles or biodegradable polymeric nanoparticles, wherein the lipid nanoparticles or biodegradable polymeric nanoparticles encapsulate the mitochondrial network enhancing agent.

As used herein, a “biodegradable polymeric nanoparticle” includes a polymer susceptible to degradation (or biodegradation) after implantation into an organism, wherein the degradation is accompanied by lowering of the polymer's molar mass. The biodegradation can proceed, for example, by hydrolysis, by contact with nasal mucus, by catalytic activity of other enzymes, or by a combination of factors including a wide variety of biological activities. The support body encloses or contains a reservoir, i.e., an open space that can be filled with a polymer matrix, such as a hydrogel. The support body can include openings and/or pores connecting the reservoir with the environment outside the support body. The reservoir can be filled with a polymer network or matrix that contains a second biodegradable polymer which forms the polymer matrix.

The biodegradable polymeric nanoparticles disclosed herein are capable of controlled rates of degradation, for example, by selecting different copolymers, by changing pore size, by selecting a thickness of the polymer, or by utilizing layers (e.g., core-shell) of polymers of varying thicknesses. When a polymer is utilized as an outer shell or hollow sheath, the term “biodegrades” can refer to cracking of an outer shell.

In an example, a hydrophilic or hydrophobic outer sheath biodegradable polymeric nanoparticle can include pores and a core-shell configuration, wherein the polymeric outer sheath around the core acts as a barrier layer to delay and further control the release profile of a therapeutic agent. The size of the pores can be adjusted, for example, by utilizing different molecular weights of polyethylene glycol (PEG) with poly(ε-caprolactone) in an outer sheath polymer. The pores can allow diffusion of a mitochondrial enhancing network agent through the outer sheath polymer to contact the olfactory mucosal epithelium. Pores can increase or decrease in size after implantation. The size of the pores can be in the range from about 0.01 μm to about 100 μm, in the range from about 1 μm to about 100 μm, in the range from about 5 μm to about 50 μm, or in the range from about 10 μm to about 30 μm. In an example, an outer sheath polymer can have an average wall thickness in the range from about 0.1 mm to about 2 mm, or in the range from about 0.1 mm to about 1 mm, or in the range from about 0.1 mm to about 0.6 mm.

The mitochondrial enhancing network agent can be provided as dissolved or suspended within the biodegradable polymeric nanoparticles as solid particles, such as nanoparticles or microparticles, within a hydrogel. The hydrogel can be an osmotic hydrogel that contains an osmotic core component, such as a dissolved or suspended osmolyte that attracts water from the biological environment to form a water-swollen polymer network that promotes the release of the mitochondrial enhancing network agent from the gel. The hydrogel can include a polymer network, a colloidal network, or a combination thereof. The hydrogel can encapsulate the payload (e.g., mitochondrial enhancing network agent) and modulate its release profile by hydrolytic swelling. Hydrogels are highly hydrophilic networks of polymer chains, sometimes found as colloidal gel networks in which water is the dispersion medium. The water-absorbing properties of the hydrogels can result from the presence of hydrophilic functional groups, for example, hydroxy (—OH), carboxylic (—COOH), amidic (—CONH—), primary amidic (—CONH2), and sulfonic (—SO3H), rather than from the osmotic pressure of the hydrogels. In hydrogel swelling, examples of relevant parameters are the swelling rate, swelling ratio, and swelling capacity, which can depend on several physiochemical factors such as the gel size, network porosity, network structure, cross-linking conditions, and cross-linking degree of the hydrogel. While typically not considered in higher molecular weight formulations, the osmolarity of hydrogels can also contribute to swelling or water uptake. The normal osmolarity of the human body is in the range from about 250 to about 350 milliosmoles. Optionally, the osmolarity of the hydrogel can be adjusted to change, for example, the swelling rate.

The biodegradable polymeric nanoparticles of the present technology include one or more biodegradable polymers selected from the group consisting of gelatin, chitosan, cellulose, cellulose derivatives, poly(ε-caprolactone) (PCL), polyethylene glycol (PEG), poly(lactic-co-glycolic acid) (PLGA), polylactic acid (PLA), polyglycolide, PEG-PLA diblock copolymer, PEG-PLGA diblock copolymer, PEG-PCL diblock copolymer, PCL-b-PEG-b-PCL co-polymer, polypropylene glycol (PPG), polyacrylic acid, polyacrylamide, poly(N-isopropylacrylamide), polyethylene oxide-polypropylene oxide-polyethylene oxide (PEO-PPO-PEO) triblock copolymer, hyaluronic acid, polyethylene oxide, polypropylene oxide, alginic acid, poly(betaaminoester) (PBAE), poly(glycolic acid) (PGA), and alginate.

In selected embodiments, the mitochondrial network enhancing nanoparticles include lipid nanoparticles including preferably liposomes, and more preferably cationic liposomes.

Cationic lipids are used in the present technology to promote cellular uptake of agents for enhancing the mitochondrial network of a cell. Cationic lipids can be incorporated into liposomes, forming cationic liposomes, or can form non-membranous lipid complexes with one or more agents thereby forming mixed micelles or other supramolecular structures. Liposomes and other lipid-containing complexes are referred to herein as “lipid nanoparticles”. Inclusion of cationic lipids in such lipid nanoparticles can promote the stabilization of an agent for delivery into a cell via endocytosis, especially if the agent is negatively charged. Cationic lipids also promote endosomal escape, thereby releasing the agent into the cytoplasm.

Any type of cationic lipid can be used in the present technology. Cationic lipids typically have a positively charged headgroup and a hydrophobic tail, and can be phospholipids or other types of lipids. Examples of cationic lipids include dioctadecyltrimethylammonium (DOTMA), dioctadecyltetramethylammonium (DOTAP), and dimethylaminoethane carbamoyl cholesterol. Various other lipids can be combined with cationic lipids in order to provide lipid nanoparticles with desired properties, as is known in the field.

Cationic liposomes can be drawn to or target the negative potential of the cellular mitochondria. The cationic liposomes include 1,2-dioleoyl-3-trimethylammonium-propane (DOTAP) as the cationic lipid for facilitating preferential mitochondrial association and further include one or more components selected from the group consisting of a neutral phospholipid for bilayer formation, cholesterol for structural integrity, and a hydrophilic polymer such as polyethylene glycol (PEG) for decreasing particle aggregation and decreasing immune clearance. In one embodiment, the composition consists of 44 wt. % dipalmitoylphosphatidylcholine (DPPC), 14 wt. % cholesterol 22 wt. % 1,2-dioleoyl-3-trimethylammonium-propane (DOTAP); and 20% polyethylene glycol (PEG).

In preferred embodiments, the zeta potential of the liposomes is greater than about +30 mV or less than about −30 mV. In alternative embodiments, the zeta potential of the liposomes is in a range from about 0 mV to about +100 mV, or from about 0 mV to about −100 mV, or from about +30 mV to about +100 mV, or from about −30 mV to about −100 mV, or from about +40 mV to about +60 mV, or from about −40 mV to about −60 mV. Preferably, the zeta potential is in a range that promotes stability and avoids aggregation of the liposomes.

The cationic liposomes are prepared by forming a lipid film, rehydrating the film with the peptide solution in water during freeze thaw cycles to form multilaminar vesicles, and probe sonicating to form liposomes. The lipid film is prepared using a cationic lipid such as DOTAP (1,2-dioleoyl-3-trimethylammonium-propane chloride salt), a stabilizer such as cholesterol, and a neutral lipid such as DPPC (1,2-dipalmitoyl-sn-7 glycero-3-phosphocholine), in a 5:3:5 molar ratio in 2 ml of chloroform using a Sigma-Aldrich ST/NS14/20 10 mL round bottom flask attached to a Rotavap (IKA works Inc. Wilmington, NC-28405, Model RV 10 C S99). Following chloroform evaporation, the films are resuspended in 1 ml of 1 mg peptide solution in di water before 5 cycles of liquid nitrogen freezing and heating in a 42 C water bath. The liposomal preparation is then probe-sonicated for 5 min on ice. The mixture is centrifuged in a Beckman-Coulter ultracentrifuge at 20,000 RPM for 15 min in Amicon (10K) centrifugal filters at 4 C to separate the peptide-encapsulated liposomes from unencapsulated peptide.

In selected embodiments, the compositions include one or more pharmaceutically acceptable excipients or carriers. The pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water, saline solutions and aqueous dextrose and glycerol solutions can also be employed as liquid excipients. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

The compositions of the present technology can be present in various formulations. Any compound and composition (and/or additional agents) described herein can take the form of solutions, suspensions, emulsion, drops, tablets, pills, pellets, capsules, capsules containing liquids, powders, sustained-release formulations, or any other form suitable for use. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the compounds and compositions can also include a solubilizing agent. Also, the compounds and compositions can be delivered with a suitable vehicle or delivery device as known in the art. Combination therapies outlined herein can be co-delivered in a single delivery vehicle or delivery device.

The formulations comprising the compounds and compositions of the present invention may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin.

In some embodiments, the composition is administered orally. In some embodiments, the administration is by absorption through epithelial or mucocutaneous linings (e.g., oral mucosa). Various delivery systems are known, e.g., encapsulation in liposomes, microparticles, microcapsules, capsules, etc., and can be used to administer. In various embodiments, the compounds and compositions of the present invention are formulated to be suitable for oral delivery. In various embodiments, the compounds and compositions of the present invention are formulated to be suitable for transmucosal delivery (see, e.g. Msatheesh, et al. Expert Opin Drug Deliv. 2012 June; 9(6):629-47, the entire contents of which are hereby incorporated by reference).

Compositions or compounds for oral delivery can be in the form of tablets, lozenges, aqueous or oily suspensions, granules, powders, emulsions, capsules, syrups, or elixirs, for example. In some embodiments, the compounds and compositions of the present invention are in the form of a capsule, tablet, patch, or lozenge. Orally administered compositions can comprise one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active capsule containing a driving compound capable of driving any compound or composition described herein is also suitable for orally administered compositions. In these latter platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be useful. Oral compositions can include standard excipients such as mannitol, lactose, starch, magnesium stearate, sodium saccharin, cellulose, and magnesium carbonate. In one embodiment, the excipients are of pharmaceutical grade. Suspensions, in addition to the active compounds, may contain suspending agents such as, for example, ethoxylated isostearyl alcohols, polyoxyethylene sorbitol and sorbitan esters, microcrystalline cellulose, aluminum metahydroxide, bentonite, agar-agar, tragacanth, etc., and mixtures thereof.

The current technology includes a method of enhancing a mitochondrial network in a cell of a mammalian subject, and preferably a human subject. The method includes:
(a) providing the composition of the invention as described above; and
(b) administering the composition to the mammalian subject, whereby the mitochondrial network is enhanced in the cell of the mammalian subject.

The method of enhancing the mitochondrial network in the cell of the mammalian subject includes enhancing fusion of mitochondria with other mitochondria and/or with endoplasmic reticulum in the cell, and/or increasing a size or density of the mitochondrial network in the cell. Such enhancement results in one or more of increased energy production, reduced reactive oxygen species (ROS) production, reduced nuclear DNA damage, reduced mitochondrial DNA damage, increased cell survival, increased protein folding efficiency, reduced protein misfolding, reduced accumulation of misfolded proteins, increased autophagy, increased mitophagy, and reduced apoptosis. The enhancement of the cellular mitochondrial network in the cell of the mammalian subject also results in reducing fission of mitochondria in cells of the mammalian subject.

The methods of the present technology can be used for the treatment of human subjects suffering from has a neurological disease or disorder, a disease or disorder related to mitochondrial dysfunction, a metabolic disease or disorder, an accelerated aging disorder, a progeroid syndrome, an MFN related disorder, Marie-Charcot Tooth disease, an autoimmune disease, such as, for a non-limiting example, Lupus, or any combination thereof. The neurological disease or disorder includes a neurodegenerative disease. The neurodegenerative disease includes Alzheimer's disease, Parkinson's disease, Huntington's disease, amyotrophic lateral sclerosis (ALS), spinal muscular atrophy, a prion disease, and combinations thereof, the disease or disorder related to mitochondrial dysfunction includes a disease related to decrease of mitochondrial energy capacity, Charcot-Marie-Tooth disease type 2A (CMT2A), a mental health condition showing mitochondrial dysfunction, a genetic mitochondrial disease, mitochondrial encephalomyopathy, lactic acidosis and stroke-like episodes (MELAS), a secondary mitochondrial dysfunction due to another condition or disease, and combinations thereof. The metabolic disease or disorder includes diabetes, obesity, metabolic syndrome, and combinations thereof. The cardiovascular disease includes ischemia, myocardial infarction, stroke, heart failure, cardiomyopathy, and combinations thereof. The progeroid syndrome includes progeria, Hutchinson-Gilford Progeria Syndrome, Werner syndrome, and combinations thereof.

The methods of the current technology further include a method of enhancing a mitochondrial network in a cell including:
(a) providing the composition of any of the preceding claims and a culture comprising the cell; and
(b) adding the composition to a medium of the culture, whereby nanoparticles of the composition are taken up by the cell and the mitochondrial network in the cell is enhanced.

This method further includes administering the cell to a mammalian subject in need thereof. In one embodiment, the cell is an immune cell, such as a CAR-T cell.

Thus, the advantages and improvements of the use of the compositions for and methods of enhancing cellular mitochondrial network of the present invention include:
1. Increasing mitochondrial network fusion.
2. Increasing mitochondrial fusion to the endoplasmic reticulum.
3. Orienting mitochondria in an anti-apoptotic conformation, protecting against cell death.
4. Protection against the misfolded/unfolded protein response.
5. Manipulates of mitochondrial dynamics to decrease cell stress and increase total cellular adenosine triphosphate (ATP) capacity.

### Example 1: Synthesis of Cationic Liposomes Loaded with a Validated Mitofusin 2 (MFN2) Activating Peptide⁴ (SEQ ID NO:1: DIAEAVRLIMDSLHMAAR)

Example 1 shows the synthesis of nanoparticles including mitochondrial network enhancing agent according to the present technology. Nanoparticles including cationic liposomes were prepared according to previously published methods²⁰. The lipid film was prepared using DOTAP (1,2-dioleoyl-3-trimethylammonium-propane chloride salt) a cationic lipid, cholesterol (stabilizer), and DPPC (1,2-dipalmitoyl-sn-7 glycero-3-phosphocholine) a neutral lipid in a 5:3:5 molar ratio using a Sigma-Aldrich ST/NS14/20 10 mL round bottom flask attached to a Rotavap (IKA works Inc. Wilmington, NC-28405, Model RV 10 C S99). Following chloroform evaporation, the films were resuspended in 1 ml of peptide solution in di water before 5 cycles of liquid nitrogen freezing and heating in a 42 C water bath. The liposomal preparation was then probe-sonicated for 5 min on ice. The mixture was centrifuged in a Beckman-Coulter ultracentrifuge at 20,000 RPM for 15 min at 4 C to separate the peptide-encapsulated liposomes from unencapsulated peptide.

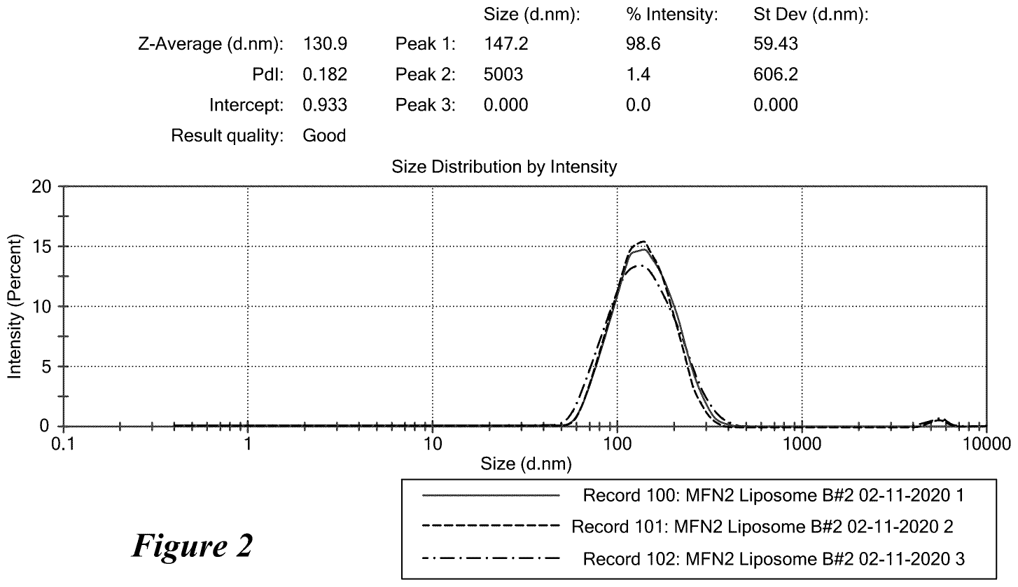

FIG. 2 shows the diameter size of the cationic liposomes loaded with the network enhancing agent including a mitofusin 2 (MFN2) peptide fragment including amino acid sequence (SEQ ID NO:1) as measured by Dynamic Light Scattering using a Malvern ZetaSizer as a function of intensity. The loaded cationic liposomes are approximately 130 nm with a narrow size distribution. Encapsulation efficiency of the peptide is approximately 70%. Zeta potential is 42 mV indicating a stable colloidal suspension.

### Example 2: Peptide Characterization with Innovagen Peptide Property Calculator and Pep-Fold Peptide Structure Prediction Server

Example 2 shows a peptide characterization and predicted structure for a mitochondrial network enhancing agent according to the present technology. Innovagen's peptide property calculator and the Pep-Fold peptide structure prediction server were used to characterize the alpha-helical mitofusin 2 (MFN2) peptide including amino acid sequence (SEQ ID NO:1), and to determine its probable peptide structure.

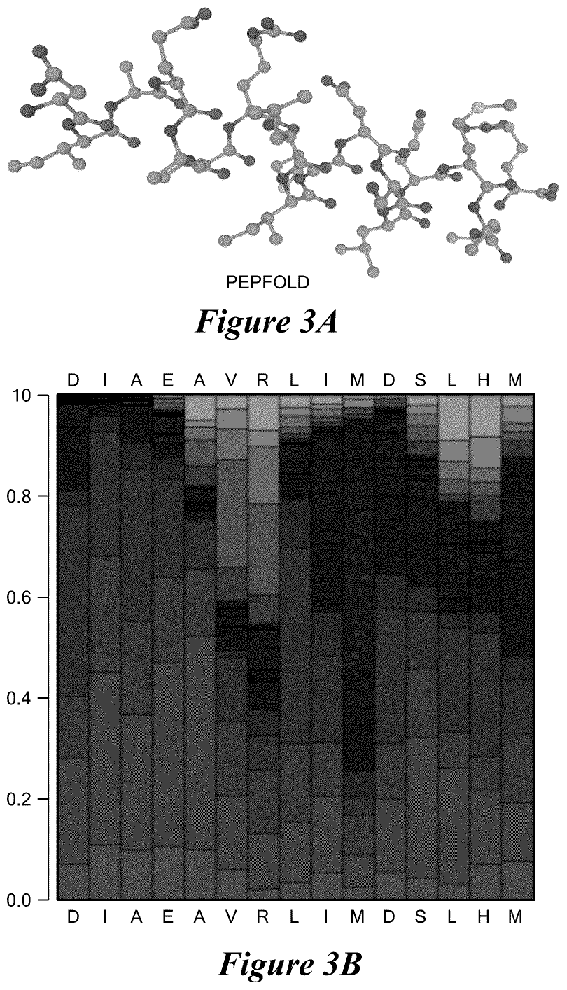

FIG. 3A shows a schematic of the peptide fragment. FIG. 3B shows a red intensity region representing helical structures; a blue intensity region representing coils; and a green intensity region representing extended regions of the peptide fragment.

The mitofusin 2 (MFN2) peptide fragment including amino acid sequence (SEQ ID NO:1) is ideal for liposomal delivery as its grand average of hydropathy (GRAVY) score is 0.461 indicating it is stable in aqueous media (the core of the liposome and the cytoplasm of the cell) and the peptide fragment has helical coils with little branching making it an ideal structure for encapsulation. The peptide fragment in solution, without the aid of a liposome would degrade rapidly in the body due to the numerous peptidases in blood and tissues.

Table 1 lists nanoparticle characterization data for blank liposome nanoparticles (Blank NP) without peptide fragments and for nanoparticles each encapsulating a mitochondrial network enhancing agent including mitofusin 2 (MFN2) peptide fragment including amino acid sequence (SEQ ID NO:1) (MiNE NP).

TABLE 1
Nanoparticle (NP) Characterization

| | Particle | Zeta | Peptide |
|---|---|---|---|
| | Diameter | Potential | Encapsulation |
| | (d · nm) | (mV) | Efficiency (%) |
| Blank NP | 161.4 ± 86.6 | 41.51 ± 3.37 | N/A |
| MiNE NP | 229.7 ± 91.2 | 38.12 ± 3.81 | 76.38 |

Composition (v/v %) = 44% DPPC, 14% cholesterol, 22% DOTAP, 20% PEG

Table 1 shows the blank liposome nanoparticles (without peptide) have an average hydrodynamic diameter of 161.4 nm, while the average particle size for nanoparticles loaded with the mitofusin 2 (MFN2) peptide fragment is 229.7 nm, respectively. The zeta potential values for both the blank and nanoparticles indicates particle stability (outside of the +30 mV to −30 mV range). The peptide encapsulation efficiency of the nanoparticles averaged 76.38%. All drug dosing was according to molarity and adjusted according to batch encapsulation efficiency.

The nanoparticles featured Table 1 are composed of 44% dipalmitoylphosphatidylcholine (DPPC), an amphipathic phospholipid ideal for bilayer formation, 14% cholesterol for structural integrity, 22% 1,2-dioleoyl-3-trimethylammonium-propane (DOTAP), a cationic lipid to facilitate preferential mitochondrial association, and 20% polyethylene glycol (PEG), a hydrophilic polymer that decreases particle aggregation and decreases immune clearance).

### Example 3: Validation and Quantification of Mitochondrial Networks in Mouse Fibroblasts (NIH3T3) Cells

The liposomes of the present technology have strong translational potential and are very similar to the lipid nanoparticles used in the current Pfizer (BNT162b2) and Moderna (mRNA1273) SARS-COV-2 vaccines. These vaccines demonstrate that lipid nanoparticles have a high safety profile, even after repeated dosing regimens²¹⁻³⁰. A second safety advantage of nanoparticle liposomal delivery is related to the large ratio of liposomal surface area relative to volume ratio to protect a patient from high systemic free drug concentrations while also protecting the drug from immune clearance^{21,31,32}. This advantage allows for a lower effective drug administration as the drug is more highly protected from immune and metabolic degradation while the lower effective dose results in lower toxicity.

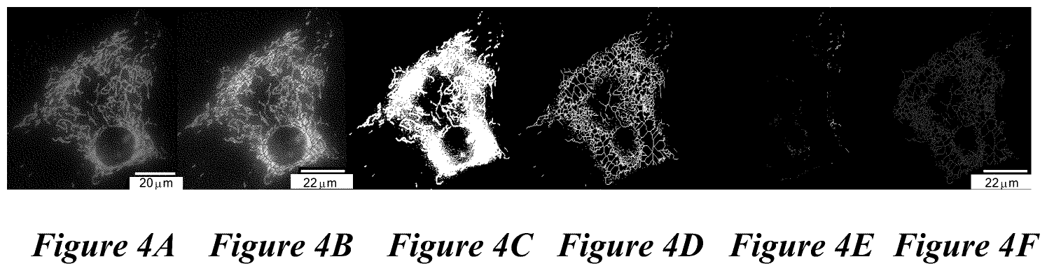

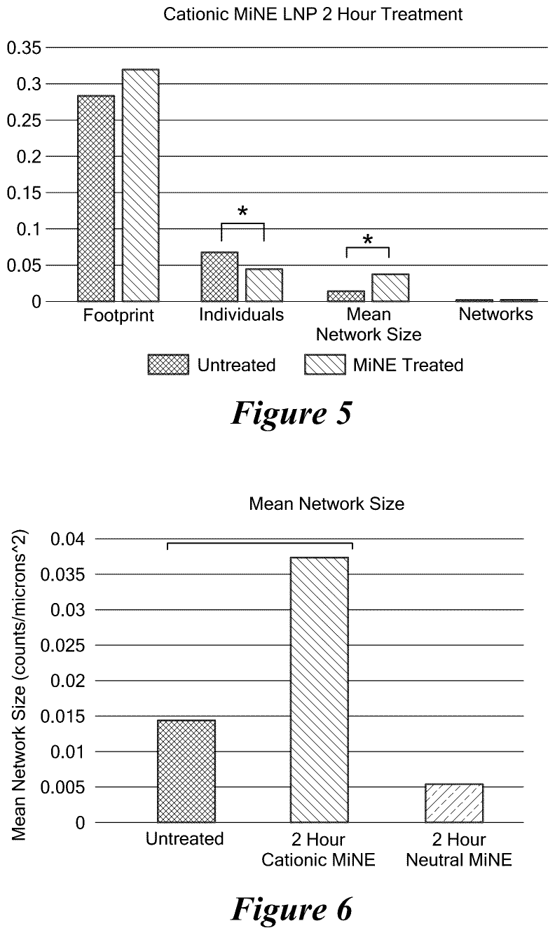

Preliminary work has validated the ability to quantify mitochondrial networks in mouse fibroblasts (NIH3T3) cells (FIGS. 4A-7) and in human cortical neurons (HCN2 cells; FIG. 11). A Keyence BZ-X710 fluorescence microscope was used to acquire the images of FIGS. 4A-F for the Mitochondrial Network Analysis (MiNA) shown in FIGS. 5-7. MiNA is an under-utilized, superior tool for measuring mitochondrial dynamics³³. Alternative methods for measuring mitochondrial dynamics rely on methods that do not mimic endogenous events. The most common method for assessing mitochondrial dynamics involves the use of polyethylene glycol to fuse two cell populations with differentially labeled (red and green) mitochondria and the observation of co-localization of the labeling³⁴. The result of this method is the creation of a hybrid cell, and this technique is not actually evaluating the mitochondrial dynamics within one cell because it is assessing the fusion of mitochondria as the result of two cells fusing together (which is not an endogenous event).

In Example 3, mouse fibroblast (NIH3T3) cells were stained with Mitotracker™ Green, and fluorescent microscopy was performed using the 60× objective of a Keyence BZ-X710 fluorescence microscope, as shown in FIGS. 4A-F. Images were processed using mitochondrial network analysis (MiNA). The scale bar in the first panel of FIG. 4A is 20 μm and in the last panel of FIG. 4F is 22 μm. As demonstrated in FIG. 4A-F, MiNA involves a series of image processing steps to obtain the mitochondrial network parameters shown in FIGS. 5-6, and 8. MiNA requires stained mitochondria in a fluorescent channel (Mitotracker™ Green (FIG. 4A); a second image and overlay on the first image (FIG. 4B) and the corresponding brightfield third image and overlay on the first image (FIG. 4C). The MiNA workflow includes converting images to a binary image (FIG. 4D) and skeletonizing the image (FIG. 4E) and then performing three-dimensional analysis (FIG. 4F). Mathematical expressions from the toolset are used to calculate individual mitochondria and mitochondrial network parameters (FIG. 5-7) from the three-dimensional mitochondrial skeleton (FIG. 4F)³³. Due to differences in cell sizes, all MiNA data is normalized to the square micron area.

MiNA analysis demonstrates that treatment with liposomes loaded with mitofusin 2 (MFN2) peptide fragment (MiNE NPs) did not increase the mitochondrial footprint corresponding to the total individual and networked mitochondria relative to untreated cells (FIG. 5, first column). Treatment with the MiNE NPs promoted a significant decrease in the number of individual mitochondria (FIG. 5, second column), and a significant increase in the size of existing networks (FIG. 5, third column). Treatment with the MiNE NPs did not promote a significant increase in the number of networks (FIG. 5, fourth column). Treatment with the MiNE NPs promoted substantially “growing” existing mitochondrial networks and building their size.

Mitochondrial network analysis of mouse fibroblast (NIH3T3) cells was performed by comparing untreated cells (purple) with treated cells (green) after two hours of treatment with the mitochondrial network enhancing nanoparticles prepared as described in Example 1.

FIG. 5 shows the Footprint, Individuals, Mean Network Size, and Networks columns corresponding to the untreated and treated cells' respective total mitochondrial footprints including the total number of individual and networked mitochondria, total number of individual mitochondria, size of the networked mitochondria, and total number of mitochondria networks. Data was normalized to area (square microns).

The Footprint columns of FIG. 5 demonstrate that treatment with the mitochondrial network enhancing nanoparticles did not increase the total number of individual and networked mitochondria of the treated cells relative to the untreated cells (see Footprint columns). The FIG. 5 Individuals columns show that the number of individual mitochondria significantly decreased following treatment, and the FIG. 5 Mean Network columns show that the size of the mitochondrial networks increased following treatment. The FIG. 5 Networks columns show that the number of mitochondrial networks was not affected by treatment. Thus, FIG. 5 shows that treatment with the mitochondrial network enhancing nanoparticles resulted in the substantial “growth” of the size or breadth of existing mitochondrial networks through the binding of individual mitochondria to the existing mitochondrial networks.

### Example 4: Comparison of Cationic and Neutral Liposome Nanoparticles Each Encapsulating Mitochondrial Network Enhancing Agent

The effect of the use of cationic versus neutral liposome nanoparticles in the present technology was examined in Example 4. Mitochondrial network analysis of mouse fibroblast (NIH3T3) cells was performed comparing untreated cells (purple), and cells treated with cationic and neutral liposomes each loaded with mitofusin 2 (MFN2) peptide fragment including amino acid sequence (SEQ ID NO:1). The liposomes were prepared substantially as discussed in Example 1. The treated cells were subjected to a two-hour treatment period. FIG. 6 shows the results of this example. The cells experiencing a cationic liposome treatment are labeled with a green color and the cells experiencing a neutral liposome treatment are labeled with an orange color. Data is normalized to area (square microns).

Mitochondria are the most negative organelle (−170 mV) within the cell. Cationic liposomes were used or exploited to achieve mitochondrial localization of the mitofusin MFN2 peptide fragment for binding to the mitochondrial mitofusin proteins.

FIG. 6 shows that treatment with the cationic liposomes encapsulating the mitofusin (MFN2) peptide fragment resulted in a significantly increase in the size of mitochondrial networks relative to untreated cells and to cells treated with neutral liposomes encapsulating the same peptide fragment. Further, the treatment of cells with the neutral liposomes did not result in a significant alteration in the size of the cellular mitochondrial network relative to the cells which were untreated.

### Example 5: Release Kinetics of Cationic and Neutral Liposome Nanoparticles Encapsulating Mitochondrial Network Enhancing Agent

The effect of the use of cationic versus neutral liposome nanoparticles on the agent release kinetics of the present technology was examined in Example 5. The ex vivo rate of release of the mitofusin 2 (MFN2) peptide fragment including amino acid sequence (SEQ ID NO:1) solution from cationic and neutral encapsulating liposomes in phosphate-buffered saline (PBS) over a time period in a range of 30 minutes to 12 hours at 37° C. was measured;

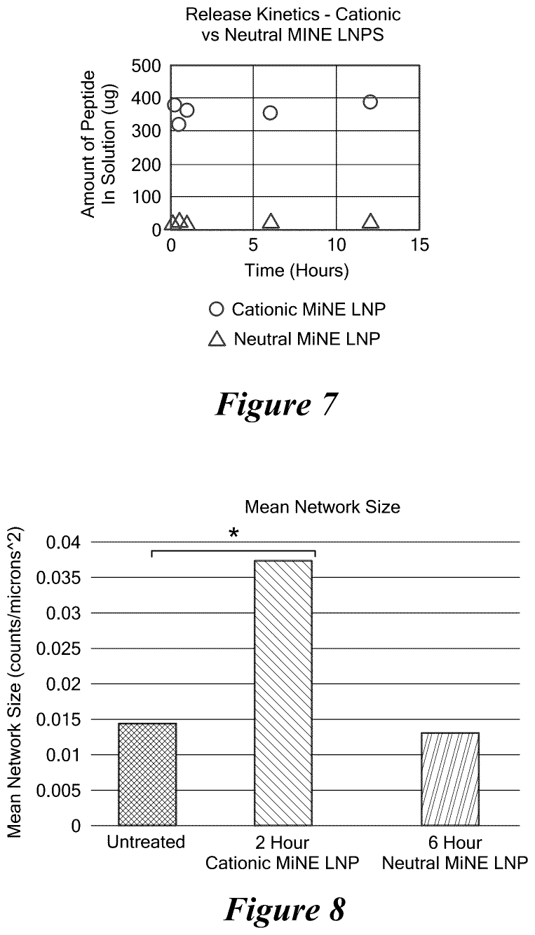

FIG. 7 demonstrates that more peptide (an average of 362.38 ug) was released from the cationic liposomes in comparison to the neutral liposomes (average of 24.22 ug) over the course of 30 minutes to 12 hours.

### Example 6: Mean Mitochondrial Network Size as a Function of Treatment Time

The effect of treatment with the composition of the present technology on mean mitochondrial network size as a function of time was examined in Example 6. The mean mitochondrial network size of untreated mouse fibroblast (NIH3T3) cells and NIH3T3 cells treated for 2 hours and for 6 hours with cationic liposomes encapsulating mitofusin 2 (MFN2) peptide fragment including the amino acid sequence (SEQ ID NO:1) was compared. The data for this example is shown in FIG. 8. The data is normalized to area (square microns). FIG. 8 shows that the ability of treatment with the cationic liposomes to promote increased mitochondrial network size occurred rapidly within the first two hours and declined after six hours of treatment in stationary cell cultures.

### Example 7: Evaluation of Mitochondrial-Endoplasmic Reticulum Fusion

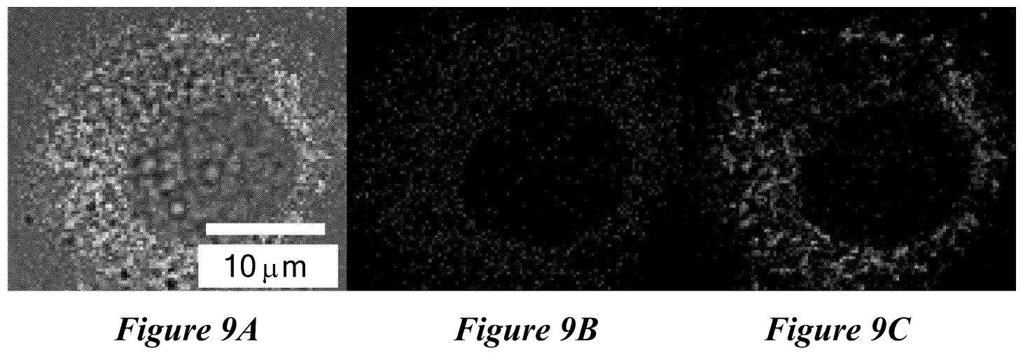

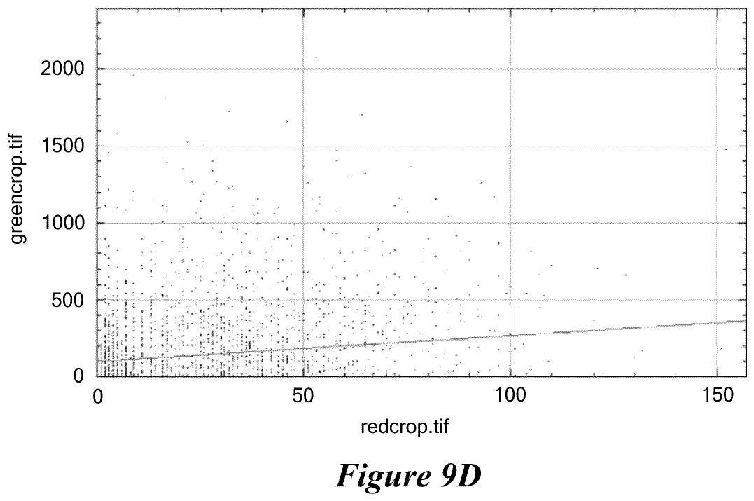

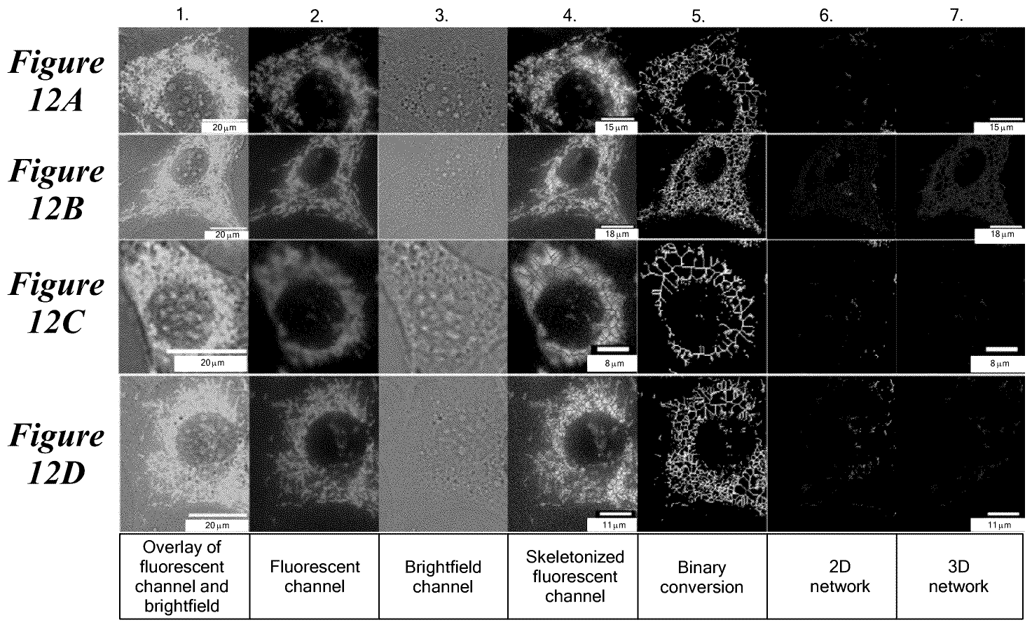

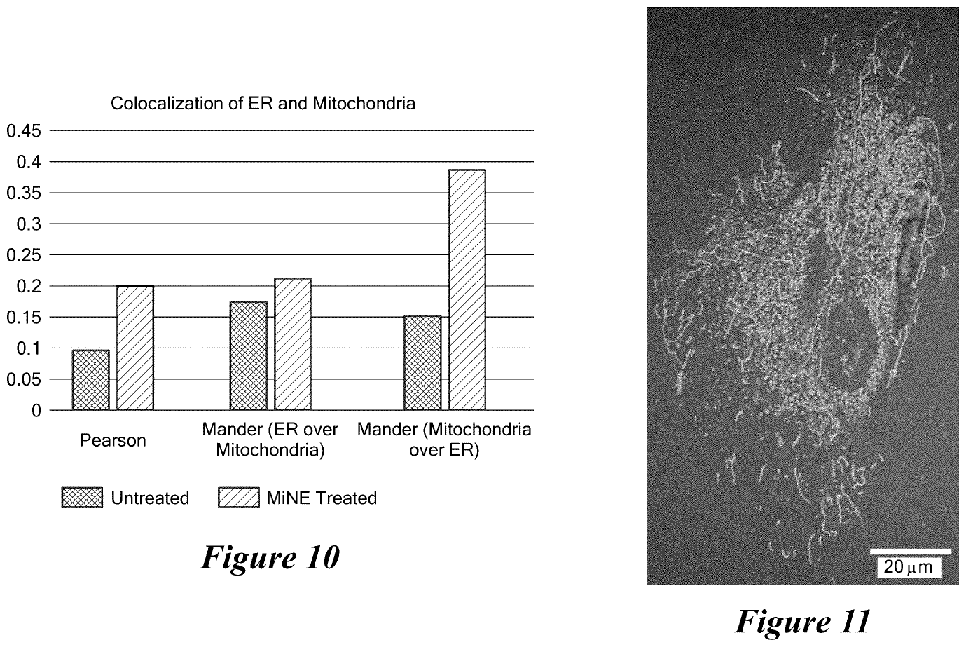

The effect of treatment with the composition of the present technology on mitochondrial-endoplasmic reticulum fusion was evaluated in Example 7 through the use of the co-localization studies shown in FIGS. 9A-9C and 10.

FIGS. 9A-9C show three fluorescence images of endoplasmic reticulum and mitochondria overlayed on top of a mouse fibroblast (NIH3T3) cell, where FIG. 9A shows a grayscale image of a cell, FIG. 9B shows the endoplasmic reticulum in red, and FIG. 9C shows the mitochondria in green. FIG. 9D shows a cytofluorogram which shows pixel co-localization of mitochondrial staining (green) and endoplasmic reticulum staining (red) using JACoP software.

FIG. 10 shows co-localization of endoplasmic reticulum and mitochondria for untreated (purple) mouse fibroblast (NIH3T3) cells and NIH3T3 cells (green) treated with cationic liposome nanoparticles loaded with mitofusin 2 (MFN2) peptide fragment including amino acid sequence (SEQ ID NO: 1), based on Pearson coefficient and Mander's coefficients of endoplasmic reticulum and mitochondria fluorescence channels, where all values are fractions of 1.

The results shown in FIGS. 9A-9C and 10 that treatment of NIH3T3 cells with cationic liposome nanoparticles loaded with mitofusin 2 (MFN2) peptide fragment including amino acid sequence (SEQ ID NO: 1) result in an increase of mitochondrial and endoplasmic reticulum co-localization.

### Example 8: Evaluation of Enhancing a Mitochondrial Network in Human Cortical Cells

A preliminary mitochondrial network evaluation of human cortical neurons after treatment ex vivo with a composition for enhancing cellular mitochondrial networks according to the present technology was conducted in Example 8. FIG. 11 shows the mitochondrial networks of human cortical neurons stained with Mitotracker™ Green in a fluorescent channel, imaged at 60× magnification, after treatment with cationic liposomes loaded with Mitofusin 2 (MFN2) peptide including amino acid sequence (SEQ ID NO:1).

In the above examples 1-8, the following cell cultures and methods were used:

Cell Culture—Mouse fibroblast NIH3T3 fibroblast cells were obtained from the American Type Culture Collection (ATCC). Cells were incubated at 5% CO2, 37 degrees C., in Dulbecco's Modified Eagle Medium supplemented with 10% Fetal Bovine Serum and 1% Penicillin-Streptomycin-amphotericin-B mixture. Cells were cultured to standard practices.

Lipid Nanoparticle Formulation—Lipid Nanoparticles (LNPs) were prepared in accordance with established methods (8). The viability of lipid nanoparticles was characterized by encapsulation efficiency, size, and zeta potential (as an indicator of stability). Encapsulation efficiency was measured using a Nanodrop mediated standard curve while size and zeta potential were obtained through the use of a Zeta Sizer.

Cell Dosing, Staining, and Imaging—Cells were seeded on 30 mm dishes and lipid nanoparticles were administered at a concentration of 10 mM, supplemented with complete DMEM solution, and incubated. After the incubation period had concluded, treatment was removed. Two-hour timepoints were conducted for neutral and cationic lipid nanoparticles and a six hour timepoint was conducted for cationic lipid nanoparticles. Post treatment, mitochondria were stained with MitoTracker™ Green (MG) Fluorescent Dye. 100 nM-200 nM concentration of (MG) diluted in DMEM solution was administered to cells over a period of 10-20 minutes. The endoplasmic reticulum was stained with ER-Tracker Red (BODIPY TR Glibenclamide) (ERTR) at a 1 uM concentration of ERTR and administered to cells over a period of 15 minutes.

Image Analysis—Mitochondrial analysis was performed through the utilization of the Mitochondrial network Analysis Toolset (MINA) on ImageJ. Slight adjustments to the documented procedure were made to optimize mitochondrial network data acquisition. Steps of image analysis are provided in text.

### Example 9: Quantification of MiNE NP Induced Changes in Mitochondrial Networks in Mouse Fibroblast NIH3T3 Cells

Mitochondrial Network Analysis (MiNA) software was used to quantify MiNE NP induced changes in mitochondrial networks in NIH3T3 cells⁴⁶. The software was developed as a compatible macro to run through NIH Image J⁴⁶. A series of image processing steps are conducted to generate a three-dimensional (3D) skeleton of mitochondrial networks⁴⁶. The 3D image is then analyzed to determine nine parameters including the number of individual mitochondria, the number of networks, the network size (number of branches), and the mitochondrial footprint⁴⁶. The mitochondrial footprint accounts for the flaws of the mathematical expression and is a quantification of the binary signal of fluorescence⁴⁶. Prior studies validated the application of this method⁴⁷.

FIGS. 12A-D show microscopy and mitochondrial network quantification of untreated and

NIH3T3 Cells treated with MiNE NP. Cells were stained with Mitotracker™ Green, fluorescent microscopy was performed using the 60× objective of a Keyence BZ-X710 microscope. Images were processed using mitochondrial network analysis (MiNA). Rows: (A) Untreated Cells, (B) MiNE NP treated cells, (C) Blank NPs, (D) MFN2 Peptide in Solution. Columns: (1) represents an overlay of the green fluorescent channel with a scalebar of 20 M (2) and the brightfield image (3). Columns 4-7 are MiNA processing of the stained mitochondrial skeleton (4), binary conversion (5), 2D network analysis (6), and 3D network quantification (7). An n=20 was used for each group.

In FIGS. 12A-D, the 60× objective of a Keyence BZ-X710 microscope was used to acquire brightfield and fluorescent images of NIH3T3 cells stained with Mitotracker™ Green. FIG. 12A is a representative untreated cell while FIG. 12B is a representative MiNE NP treated cell (10 uM). FIG. 12C is a representative cell treated with blank NPs while FIG. 12D is a representative cell treated with MFN2 peptide in solution. The columns are (1) the fluorescent overlay on the brightfield channel, (2) the green channel, (3) bright field, (4) skeletonized green channel, (5) binary image, (6) 2D network, and (7) 3D network. The mitochondrial footprint is measured from the binary image (5) while all other parameters are quantified from the 3D network (7). As all analysis is performed on single cells, the network parameters are normalized to square microns of cell area.

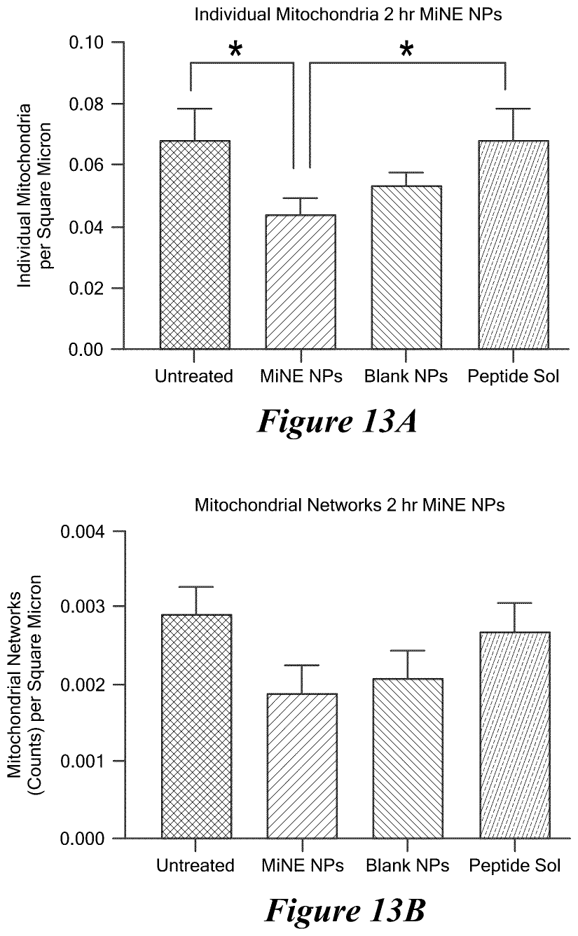

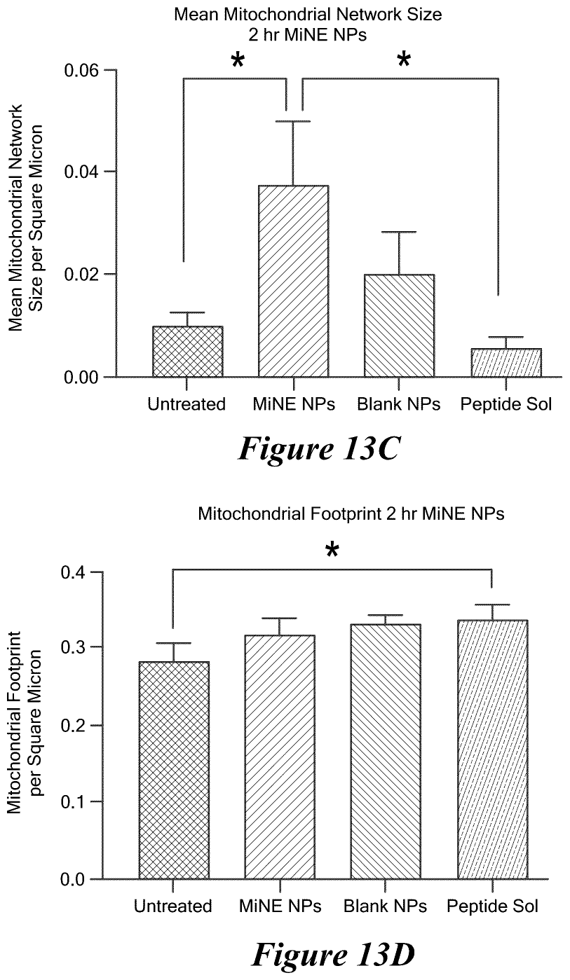

FIGS. 13A-D show mitochondrial network analysis (MiNA) for the cells featured in FIGS. 12A-D, and demonstrates that after two hours, the MiNE NPs treatment promotes a decrease in individual mitochondria and an increase in mean networks size in NIH3T3 Cells. The mitochondrial network analysis (MiNA) of 20 samples per group was performed to quantify changes induced by treatments. The footprint is the entire fluorophore staining per square microns. To account for size differences in cells, all data is normalized to the square micron area of each image. GraphPad Prism was used to conduct a Welch's t-test (n=20) and graph mean with scanning electron microscopy (SEM).

FIG. 13 thus illustrates the parameters measured from the three-dimensional network images. FIG. 13A demonstrates that treatment with MiNE NPs promotes the decrease in the number of individual mitochondria per square micron relative to untreated cells and cells treated with the MFN2 peptide in solution. FIG. 13B demonstrates that there is no significant change in the number of mitochondrial networks after MiNE NP treatment. Treatment MiNE NPs were effective in promoting the growth of existing mitochondrial networks (FIG. 13C). FIGS. 13A and 13C suggest that treatment with MiNE NPs facilitate the addition of individual mitochondria to existing network branches as the network size represents the number of branches per network. The treatment with MiNE NPs does not change the total fluorescent signal from mitochondria although the peptide solution treatment slightly increased the mitochondrial footprint. Treatment with MiNE NPs was expected to promote the increase in the number of mitochondrial networks relative to untreated cells as the process of creating a new network (fusing individual mitochondria) involves MFN2 just as building onto an existing network. It is possible that the MFN2 on existing networks is more sensitive to MFN2 activation and recruiting individual mitochondria to build branches on an existing network of fused mitochondria. Individual mitochondria may be less sensitive to MFN2 activation and more likely to join an existing network than to join with other individual mitochondria. It is also possible that the kinetics and dosing of altering mitochondrial networks were not captured after 2 hours of treatment with 10 uM MiNE NP. The direct correlation between the ability of treatment with MiNE NPs to promote a decrease in individual mitochondria and to promote an increase in the size of existing mitochondrial networks is very promising for increasing mitochondrial function. Mitochondria in network conformation are more resistant to cell death signaling and have a higher capacity for producing ATP.

### Example 10: Evaluation of MiNE Nanoparticles Increase Mitochondrial-Endoplasmic Reticulum (ER) Co-Localization

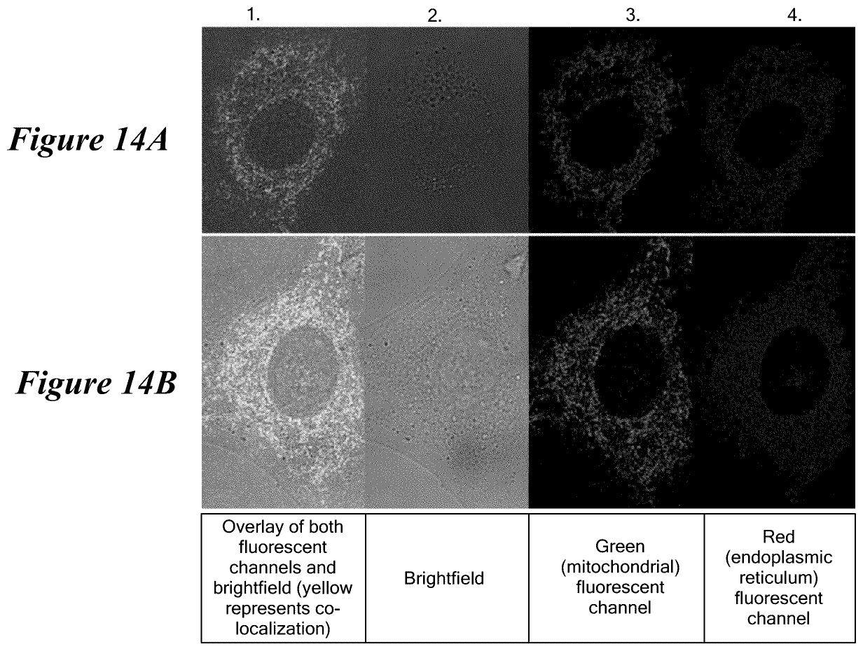

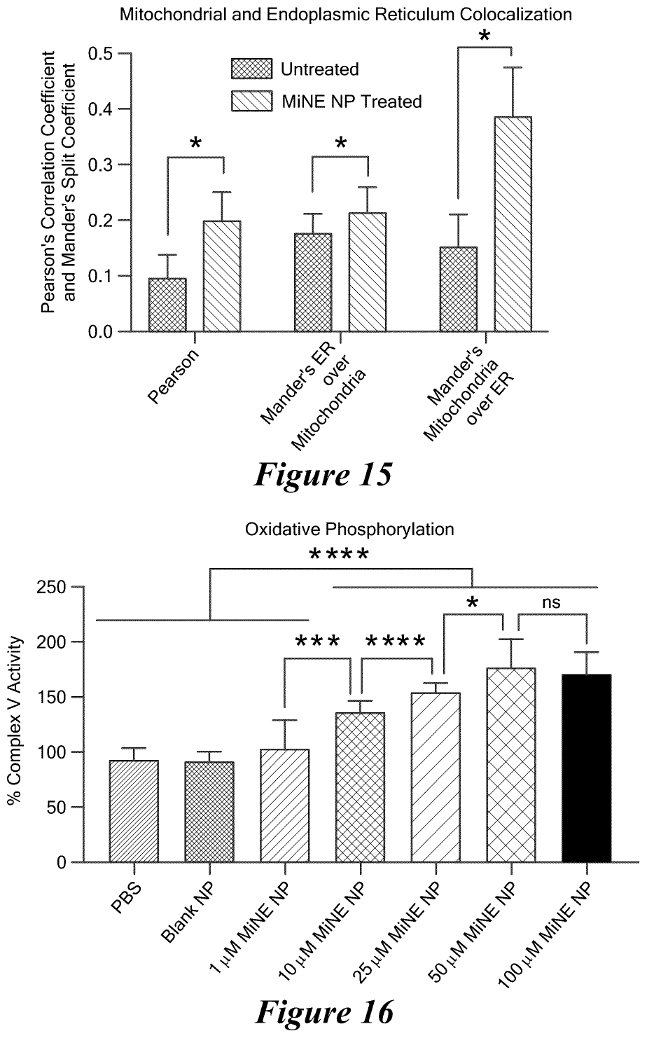

The function of mitochondria in fusing to the ER is critical for maintaining ER health and rescuing the ER from the unfolded protein response (UPR)^{33,38-41}. The ability of MiNE NPs to increase mitochondrial fusion with ER was evaluated in co-localization studies shown in FIGS. 14A-B and FIG. 15.

FIGS. 14A-B show microscopy results of mitochondrial and endoplasmic reticulum colocalization for untreated mouse fibroblast (NIH3T3) cells and NIH3T3 cells treated with liposomes loaded with mitofusin 2 (MFN2) peptide fragment including amino acid sequence (SEQ ID NO:1) (MiNE NP Treated). Cells were stained with Mitotracker™ Green and Bodipy ER Red and fluorescent microscopy was performed using the 60× objective of a Zeiss LSM 880 microscope to obtain images of untreated cells (A) and MiNE NP treated cells (B). The columns represent the (1) overlay of the three channels (yellow signal indicates co-localization), (2) bright field, (3) green mitochondrial channel, and (4) red endoplasmic reticulum (ER) channel. Representative images of n=20. Image J and the JACoP plugin (a colocalization macro) were used to process the images and three colocalization statistical values were used to determine the differences between treated and untreated groups: Pearson's correlation coefficient, Mander's split coefficients for ER over mitochondria and mitochondria over ER.

The first value analyzed, Pearson's Correlation Coefficient, determines the overlap of fluorescence in each channel without regard for intensity. Pearson's correlation coefficient is just a measure of spatial co-occurrence of fluorescence and output is expressed at values ranging from −1 to 1 with the value of −1 being perfect anticorrelation, 0 being no correlation, and 1 being perfect correlation. The value gives an effective measure of how much area colocalization exists in. The second colocalization analytic utilized is the Mander's split coefficient, which gives insight into the intensity of fluorescence for each group. Mander's output measures the fraction of intensity of the desired channel as a value from 0 to 1 in all pixels where the concurrent channel exhibits a non-zero value.

FIG. 15 shows mitochondrial and endoplasmic reticulum (ER) colocalization results for untreated mouse fibroblast (NIH3T3) cells and NIH3T3 cells treated with liposomes loaded with mitofusin 2 (MFN2) peptide fragment including amino acid sequence (SEQ ID NO:1) (MiNE NP Treated) Colocalization of untreated and MiNE NP treated cells was measured through Pearson and Mander's coefficients for pixel overlap. Data analysis for mitochondrial-ER colocalization was also conducted through ImageJ using the JACoP plugin. GraphPad Prism 9.0 (Mean and SD).

In FIG. 15, all statistical determinants of colocalization show that cells treated with MiNE NPs exhibited higher levels of colocalization compared to control cells. Higher levels of colocalization imply mitochondria and the ER occupy the same position in cell. Pearson's coefficient provides insight to the overall colocalization and shows that MiNE NP treated cells experience larger total overlap of mitochondria and ER. Both Mander's coefficients show that the intensity of fluorescence is increased in MiNE NP treated cells. This could indicate the possibility that cells are experiencing higher levels of mitochondrial-ER contact.

The accumulation of misfolded proteins and protein aggregates is a hallmark of neurodegenerative disease and a consequence of ER inadequacy. The ability of MiNE NPs to increase mitochondrial co-localization with the ER could increase ER efficiency, decrease the accumulation of misfolded proteins and, as further studies demonstrate, rescue the ER from the UPR.

### Example 11: Evaluation of MiNE Nanoparticles Increase of Oxidative Phosphorylation (OSPHOS)

FIG. 16 shows Abcam's MitoTox™ Complex V Oxidative Phosphorylation (OXPHOS) Assay results with increasing concentrations of liposomes loaded with mitofusin 2 (MFN2) peptide fragment including amino acid sequence (SEQ ID NO:1) on complex v activity in purified mitochondria. MiNE NPs significantly increase complex v activity relative to normal OXPHOS and blank nanoparticle (Blank NP) Treatment. Abcam's MitoTox™ Complex V OXPHOS Assay demonstrated that MiNE NPs and dose escalation increase Complex V activity. MiNE NPs treatments included 1 uM, 10 uM, 25 uM, 50 uM, and 100 uM. PBS was used as a negative control. n=7. From 1 uM to 50 uM MiNE NPs there was an increase in Complex V activity corresponding to the increased dose. The significance of this increase decreased with the 25 uM to 50 uM transition and seems to plateau between 50 to 100 uM indicating there may be a saturation or other plateau event. Significance was determined using Welch's t-test in GraphPad Prism 9.0 (Mean and SD).

Mitochondrial dysfunction in aging and neurodegenerative disease are associated with energy depletion^{2,8-12}. Thus, the effect of MiNE NPs on OXPHOS activity was assessed (FIG. 16) using Abcam's MitoTox™ Complex V OXPHOS Activity Assay Kit as complex v is the final ATP producing subunit of the OXPHOS machinery. The effect of increasing (1 μM-100 μM) concentrations of MiNE NPs on purified mitochondria complex v activity were evaluated continuously for 2 hours. The rate of the reaction (change in OD per minute) was determined using the data at 30 minutes and 120 minutes and converted to percent activity.

FIG. 16 shows that treatment with PBS, blank NPs, and 1 μM MiNE NPs resulted in 100% complex V activity. Treatment with 10 μM significantly increased complex v activity to an average of 136.5% while treatment with 25 μM significantly increased activity to an average of 154.2%. Although with less significance, treatment with 50 μM significantly increased activity to an average of 176.5% while there was not a significant increase with 100 μM MiNE NP treatment. MiNE NPs demonstrate a sigmodal response in complex v activity. The lack of response for 1 μM is expected to be due to the low dose of the peptide while the lack of increase at 100 μM could represent a saturation of complex v activity or the plateau of MiNE NP efficacy. The ability of MiNE NPs to increase complex v activity in purified mitochondria suggests that mitochondrial fusion/MFN2 activity could increase OXPHOS in cells, offering a new treatment avenue for increasing energy capacity in neurodegenerative disease.

### Example 12: Evaluation of MiNE Nanoparticles Protect Against the Unfolded Protein Response

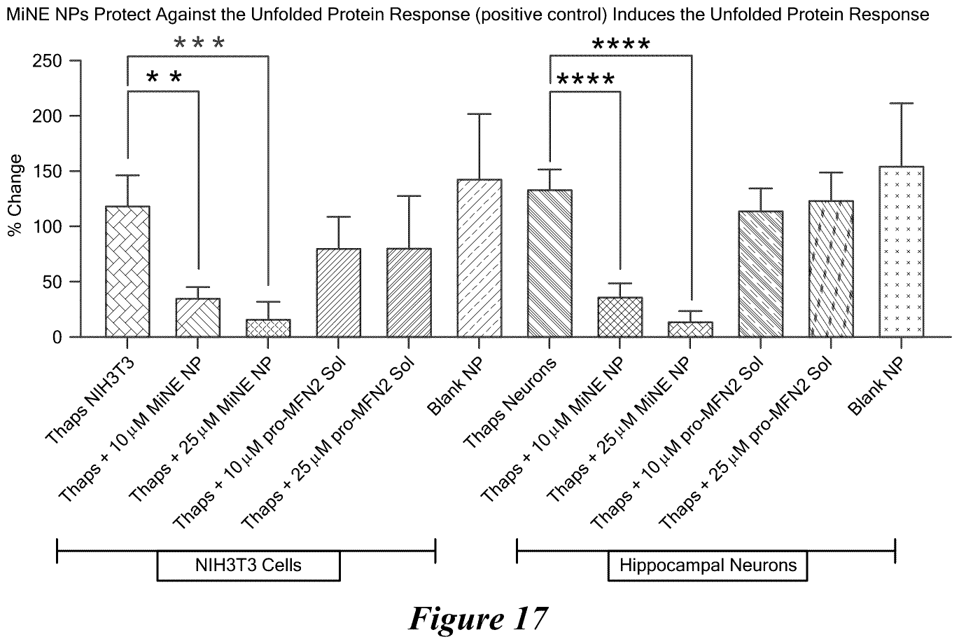

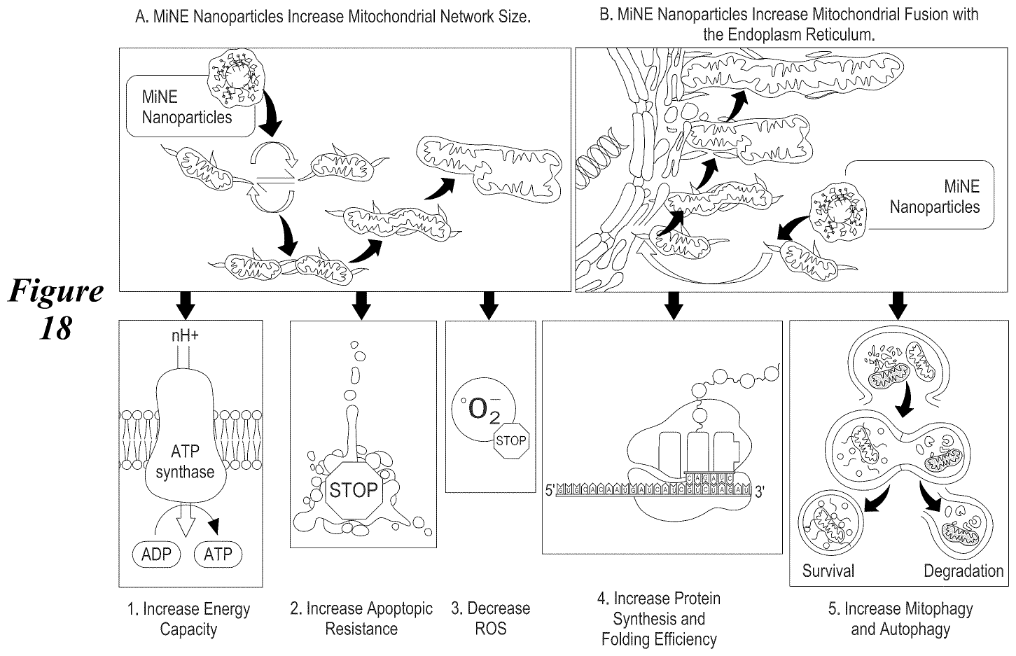

FIG. 17 shows an evaluation of the ability of the liposomes loaded with mitofusin 2 (MFN2) peptide fragment including amino acid sequence (SEQ ID NO:1) to protect against the unfolded protein response (UPR) in mouse fibroblast (NIHT3T) cells and in hippocampal neurons. FIG. 17 shows that MiNE NPs protect against the UPR. The positive control induces the UPR. The Montana Molecular UPR Assay was used to quantify ER stress and was compared to the known UPR inducer thapsigargin (Thaps). Activity was measured over 6 h of treatment and cell stressor in NIH3T3 cells (left 6 columns) and in primary hippocampal neurons (right 6 most columns). Significance is only shown for MiNE NPs. There was no significance between thapsigargin and all other treatments. Significance was determined using Welch's t-test in GraphPad Prism 9.0. n=9 with triplicate studies (Mean and SD).

Mitochondria are not the only dysfunctional organelle in neurodegenerative disease. The accumulation of misfolded and aggregated proteins is well documented in neurodegenerative disease which is associated with a level of ER deficiency³⁰⁻³². The UPR in neurodegenerative disease is under-investigated, but it has been noted to be increased in AD^{44,45}. There is a close intersection of the UPR, mitochondrial-ER fusion, mitophagy/autophagy, and cell death/survival decision points^{33,38-42}. The ability of MiNE NPs to protect against the UPR was assessed in NIH3T3 cells and in hippocampal neurons (FIG. 17). Thapsigargin (Thaps), a positive control provided with the kit, was used to induce the UPR. The UPR induced by thapsigargin co-incubated with 10 μM and 25 μM MiNE NPs and MFN2 solution was evaluated at time zero and 6 hours post treatment, with % change correlating to an increase in the UPR measured via a fluorescent reporter. In both cell lines, the MFN2 solution did not rescue cells from thapsigargin induced UPR. Over the course of 6 hours, MiNE NPs (both at 10 μM and 25 μM) significantly decreased the thapsigargin induced UPR. Interestingly, this effect was more significant in the primary hippocampal neurons than in the immortalized NIH3T3 cells. There are a plethora of possibilities for this, spanning from cellular sensitivity to the UPR to differences in mitochondrial-ER ratios. In both cell lines, blank NPs had no effect on preventing the UPR. Mitochondrial fusion to the ER has been reported to rescue the ER from the UPR^{33,38-41}. The ability of MiNE NPs to decrease the ER response to stress and prevent the induction of the UPR could slow the detrimental effects of misfolded protein accumulation in neurodegenerative disease.

### Example 13: Evaluation of Nanoparticles Potential Mechanism of Action

These preliminary studies of MiNE NPs have demonstrated that MiNE NPs can grow mitochondrial networks, increase mitochondrial-ER co-localization, increase complex v activity, and protect NIH3T3 cells and neurons from the ER stress induced UPR. As mitochondrial dysfunction and misfolded protein accumulation are central to neurodegenerative disease, MiNE NPs have strong potential as a future treatment for AD and PD.

The mitochondrial network enhancing (MiNE) nanoparticle mechanism of action is schematically illustrated in FIG. 18. FIG. 18 illustrates MiNE NPs increase mitochondrial network size (A) and this study demonstrated that this increase in network size results in (1) increased energy capacity as demonstrated by Complex V activity. It is hypothesized, that this increased mitochondrial network size will (2) increase apoptotic resistance as networked mitochondria are conformationally protective against pro-apoptotic signaling. Increased network size is also hypothesized to (3) decrease ROS due to increased mitochondrial health through the fusion-mitophagy axis. MiNE NPs also (B) increase mitochondrial to endoplasmic reticulum fusion. This study demonstrated that MiNE NPs can rescue cells from the Unfolded Protein Response (UPR) and we hypothesize (and will further test) that MiNE NPs (4) are also able to increase protein synthesis and folding efficiency. A further hypothesized outcome of increased mitochondrial-endoplasmic reticulum fusion is (5) increased mitophagy and autophagy as the fusion of these organelles in tightly coupled to cell survival after the UPR through autophagic pathways.

As illustrated in FIG. 18, thus it is further hypothesize that MiNE NPs could increase apoptotic resistance through the conformationally protective orientation of networked mitochondria which decreases pro-apoptotic Bcl2 family member accessibility to binding sites on the OMM. By increasing mitochondrial health (through increased efficiency of OXPHOS functioning and increased mitophagy), a decrease in ROS accumulation is expected to result in a decrease in mtDNA damage and cellular ROS induced damage. Through increased mitochondrial fusion with the ER, MiNE NPs are expected to increase protein synthesis and folding efficiency (under non-UPR and UPR conditions). This outcome would be transformative in the treatment of AD and PD. Through the UPR axis, MiNE NPs could potentially increase mitophagy and autophagy, increasing mitochondrial health and overall cellular survival. MiNE NPs are the first treatment for neurodegenerative disease to manipulate mitochondria and the ER. This organelle level approach could calm the perfect storm of mitochondrial dysfunction that contributes to the development and progression of neurological disease.

In Examples 9-13, the following materials and methods were used:

### Cell Culture

NIH3T3 fibroblast cells were obtained from American Type Culture Collection (ATCC). Cells were incubated at 5% CO₂, 37° C., in Dulbecco's Modified Eagle Medium supplemented with 10% Fetal Bovine Serum and 1% Penicillin-Streptomycin-amphotericin-B mixture. NIH3T3 cells were maintained with media changes every 3 days. Cell passaging was performed when cells reached 100% confluency, typically 5-7 days from the previous passage. Cell passaging was performed according to standard cell biology procedures, utilizing Trypsin-EDTA as the dissociation agent. Gibco™ Primary Rat Sprague Dawley Hippocampal Neurons were purchased from Fisher Scientific. Cells were resuspended in complete Neurobasal™ plus medium supplemented with 25 uM GlutaMAX™ I supplement and B-27™ plus supplement and seeded directly into 96 well plates at 20,000 cells per well.

### Nanoparticle Formulation

NPs were prepared in accordance with established methods⁴⁸. Relevant materials used in the formation of lipid films include: DPPC (1,2-dipalmitoyl-sn-7 glycero-3-phosphocholine), DOTAP (1,2-dioleoyl-3-trimethylammonium-propane chloride salt), PEG-2000, cholesterol, and chloroform. Upon combination of relevant materials, 1 mL of the solution was added to a 10 mL round bottom flask and attached to a Rotavap (IKA works Inc. Wilmington, NC-28405, Model RV 10C S99). The flask rotated at 200 rpm in a 37° C. water bath while a vacuum pressure was applied for 5 minutes. Post-Rotavap, full solvent evaporation was ensured by placing the flask in a vacuum desiccator chamber for 24 hours. After complete evaporation, the lipid film was rehydrated with 1 mL of solution. For MiNE NPs, a solution of 1 mg of a validated MiNE peptide (DIAEAVRGIMDSLHMAAR), dissolved in 1 mL of deionized water was used to rehydrate the lipid film²¹. For blank NPs, the film was rehydrated with 1 mL deionized water. Flasks were then put through a freeze-thaw-vortex cycle. Flasks were submerged in liquid nitrogen until frozen, thawed above the lipid transition temperature using the Rotavap at 200 rpm in a 42° C. water bath for 5 minutes, then vortexed for 30 seconds. This process was repeated 5 times. The remaining solution was extracted from the round bottom flask and transferred to a 15 mL conical tube. This tube was immersed in an ice bucket and sonicated for 5 minutes, using a cycle of two seconds of sonication followed by two seconds of rest. NPs were transferred to Amicon® ultra-membrane filter tubes with a molecular weight cutoff of 100 kDA. The tubes were centrifuged at 20,000 rpm for 15 minutes, allowing for NP collection.

Encapsulation efficiency of the MiNE NPs was determined using the Nanodrop One spectrophotometer (ND). To achieve this, a known set of standards was read on the ND, allowing for the generation of a standard curve. Each concentration was measured in triplicate, then averaged to determine each data point on the curve. The NP supernatant, located in the basin of the filter tube, was extracted and measured in triplicate as well. Using the standard curve equation previously generated, the concentration of MiNE peptide present in the supernatant was interpolated. To determine encapsulation efficiency the supernatant concentration was subtracted from the total amount of peptide introduced to the NPs, giving the amount of peptide successfully encapsulated during the formulation process. For determination of encapsulation efficiency the following formula was used: (amount of peptide encapsulated (μg)/total peptide present in system (μg))*100.

Size and zeta potential were measured using a Malvern Pan Analytical Zeta Sizer. To measure particle size, 1-2 μL of formulated NPs were added to 1 mL of deionized water in the ZS appropriate cuvette. Upon placing the cuvette into the ZS, the relevant ZS size protocols were run. This process was repeated for measuring the zeta potential. It is important to note that NPs containing a zeta potential that fall within of the −30 to +30 mV range should be considered unstable and are at risk for precipitating in a short time interval and cannot be stored for prolonged periods. Multiple NP values were averaged to characterize NPs.

### Cell Dosing, Staining and Imaging

For intracellular imaging, relevant cell lines were seeded on 35 mm glass bottom dishes (Ibidi) at a density of 100,000 cells/dish. Prepared NPs were administered directly to cells at a concentration of 10 uM and supplemented with 2 mL of complete DMEM solution, sufficient to fully cover the cell monolayer. Dishes were then incubated for the experimentally relevant time period. After the incubation period concluded, all media was removed, and cells were refreshed with 2 ml of complete DMEM solution.

Intracellular mitochondria were identified through fluorescence microscopy. Post NP treatment, mitochondria were stained with MitoTracker™ Green FM (MG) Fluorescent Dye. Staining solutions were created by diluting MG in DMEM solution to 100 nM. Solutions were administered directly to cells at 2 mL volumes, then incubated for 15 minutes. The MG/DMEM solution was removed from dishes and cells were refreshed with 2 mL fresh DMEM for imaging. Microscopy for mitochondrial networks in the NIH3T3 cell line was conducted on the Keyence Microscope using the green fluorescence channel and brightfield.

To quantify ER presence in cells, cells were stained with ER-Tracker™ Red (BODIPY™ TR Glibenclamide, for live cell imaging) dissolved in DMEM to a final concentration of 1 uM. In the NIH3T3 cell lines plated in 35 mm dishes, DMEM media was replaced with 2 mL of ER staining media and incubated for 15 minutes. Following incubation, staining media was removed, and cells were refreshed with 2 mL complete DMEM solution. Subsequently, cells were imaged using the Zeiss LSM 880 (inverted) confocal laser scanning microscope to obtain fluorescence images. Mitochondria in the NIH3T3 cell line stained (following previously stated protocol) in conjunction with ER were also imaged using the Zeiss LSM 880.

### The Unfolded Protein Response and Oxidative Phosphorylation Assay

At the level of the endoplasmic reticulum, cell stress can lead to the unfolded protein response (UPR) which can lead to the accumulation of misfolded proteins and cell death, similar to the occurrence of misfolded proteins in neurodegenerative disease. To quantify the efficacy of MiNE NPs to protect mitochondria from the UPR, Montana Molecular's Green Fluorescent Cell Stress Sensor Assay was used. Protocols were conducted according to the recommended assay user manual provided by Montana Molecular. The UPR was measured using Montana Molecular's green, fluorescent cell stress sensor kit (WO2019014072A1). A BacMam fluorescence biosensor that detects IRE1 (an ER stress sensor) mediated splicing of XBP1 RNA, which is a transcription factor mediated by the UPR. In summary, cell lines were transduced with provided Cell Stress Upward Sensor BacMam and sodium butyrate, plated in a 96 well plate, then incubated overnight. The following day, cells were refreshed with culture media and treatments were added to the plate, then incubated. All stressed groups were also treated with the cell stressor Thapsigargin. After incubation was complete, fluorescent intensity was measured using a Biotek Synergy multiplate reader. Cells experiencing cell stress exhibited higher fluorescence values (XBP1 splicing). Data analysis and representation was conducted using Microsoft Excel and Graphpad Prism.

The final protein complex of the OXPHOS machinery is complex V, also known as ATP synthase, which phosphorylates ADP to ATP. The MitoTox™ Complex V OXPHOS Activity Assay Kit was used to determine the effect of MiNE NPs on OXPHOS in extracted bovine cardiac mitochondria. Dose response studies were performed and results were obtained using a Biotek HT synergy plate reader (OD 340 nm).

### Data Analysis

Mitochondrial analysis was performed through the utilization of the Mitochondrial Network Analysis Toolset (MINA) on ImageJ⁴⁶. Images were initially uploaded into ImageJ and split into individual color channels. Two preprocessing steps were applied to the green channel (mitochondria): the unsharp mask filter and enhancing the local contrast. Post image processing, the green and brightfield channels were merged into one image. The cell of interest was isolated and saved. MINA was used at this step on the isolated cell's green channel to identify the mitochondrial footprint. Following this step, the channel was skeletonized and put through ImageJ's skeleton 2D/3D analysis. Using the analyzed channel, MINA was executed to collect the additional data: individual mitochondria (counts), number of mitochondrial networks (counts), and mean network size (counts).

Data analysis for mitochondrial-ER colocalization was also conducted through ImageJ. Cell images were processed according to previously stated image preprocessing steps, then the JACoP plugin (a colocalization macro) was run on the images to determine colocalization of the mitochondrial and endoplasmic reticulum channels. Graphpad Prism was used to calculate the significance of all experiments.

### SEQUENCE LISTING INCORPORATION BY REFERENCE

The content of the XML file of the sequence listing named “Sequence-Listing-1Dec2025-19815-1044.xml”, having a size of 1939 bytes and a creation date of 1 Dec. 2025, and electronically submitted via Patent Center on 2 Dec. 2025, is incorporated herein by reference in its entirety.

### REFERENCE LIST 1

1 Trani, J. P. et al. Mesenchymal stem cells derived from patients with premature aging syndromes display hallmarks of physiological aging. Life science alliance 5, e202201501, doi:10.26508/lsa.202201501 (2022).
2 Filadi, R., Pendin, D. & Pizzo, P. Mitofusin 2: from functions to disease. Cell Death & Disease 9, 330, doi:10.1038/s41419-017-0023-6 (2018).
3 Scorrano, L. Keeping mitochondria in shape: a matter of life and death. European Journal of Clinical Investigation 43, 886-893, doi:10.1111/eci.12135 (2013).
4 Franco, A. et al. Correcting mitochondrial fusion by manipulating mitofusin conformations. Nature 540, 74-79, doi:10.1038/nature20156 (2016).
5 Landes, T. & Martinou, J.-C. Mitochondrial outer membrane permeabilization during apoptosis: The role of mitochondrial fission. Biochimica et Biophysica Acta (BBA)—Molecular Cell Research 1813, 540-545, doi.org/10.1016/j.bbamcr.2011.01.021 (2011).
6 Renault, Thibaud T. et al. Mitochondrial Shape Governs BAX-Induced Membrane Permeabilization and Apoptosis. Molecular Cell 57, 69-82, doi:10.1016/j.molcel.2014.10.028 (2015).
7 Chen, H., McCaffery, J. M. & Chan, D. C. Mitochondrial fusion protects against neurodegeneration in the cerebellum. Cell 130, 548-562, doi:10.1016/j.cell.2007.06.026 (2007).
8 Yuan, H. et al. Mitochondrial fission is an upstream and required event for bax foci formation in response to nitric oxide in cortical neurons. Cell death and differentiation 14, 462-471, doi:10.1038/sj.cdd.4402046 (2007).
9 Karbowski, M. et al. Spatial and temporal association of Bax with mitochondrial fission sites, Drp1, and Mfn2 during apoptosis. The Journal of cell biology 159, 931-938, doi:10.1083/jcb.200209124 (2002).
10 Neuspiel, M., Zunino, R., Gangaraju, S., Rippstein, P. & McBride, H. Activated mitofusin 2 signals mitochondrial fusion, interferes with Bax activation, and reduces susceptibility to radical induced depolarization. The Journal of biological chemistry 280, 25060-25070, doi:10.1074/jbc.M501599200 (2005).
11 Frank, S. et al. The role of dynamin-related protein 1, a mediator of mitochondrial fission, in apoptosis. Developmental cell 1, 515-525, doi:10.1016/s1534-5807 (01) 00055-7 (2001).
12 Bravo, R. et al. Endoplasmic Reticulum and the Unfolded Protein Response: Dynamics and Metabolic Integration. International review of cell and molecular biology 301, 215-290, doi:10.1016/B978-0-12-407704-1.00005-1 (2013).
13 McGrath, E. P. et al. The Unfolded Protein Response in Breast Cancer. Cancers 10, 344 (2018).
14 Oslowski, C. M. & Urano, F. Measuring ER stress and the unfolded protein response using mammalian tissue culture system. Methods in enzymology 490, 71-92, doi:10.1016/B978-0-12-385114-7.00004-0 (2011).
15 Peñaranda Fajardo, N. M., Meijer, C. & Kruyt, F. A. E. The endoplasmic reticulum stress/unfolded protein response in gliomagenesis, tumor progression and as a therapeutic target in glioblastoma. Biochemical Pharmacology 118, 1-8, doi.org/10.1016/j.bcp.2016.04.008 (2016).
16 Bravo-Sagua, R. et al. Cell death and survival through the endoplasmic reticulum-mitochondrial axis. Curr Mol Med 13, 317-329, doi:10.2174/156652413804810781 (2013).
17 Milisav, I., Šuput, D. & Ribarič, S. Unfolded Protein Response and Macroautophagy in Alzheimer's, Parkinson's and Prion Diseases. Molecules 20, doi:10.3390/molecules201219865 (2015).
18 Bravo, R. et al. Increased ER-mitochondrial coupling promotes mitochondrial respiration and bioenergetics during early phases of ER stress. Journal of Cell Science 124, 2143-2152, doi:10.1242/jcs.080762 (2011).
19 Bravo, R. et al. in International Review of Cell and Molecular Biology Vol. 301 (ed Kwang W. Jeon) 215-290 (Academic Press, 2013).
20 Pawar, G. N., Parayath, N. N., Nocera, A. L., Bleier, B. S. & Amiji, M. M. Direct CNS delivery of proteins using thermosensitive liposome-in-gel carrier by heterotopic mucosal engrafting. PLOS ONE 13, e0208122, doi:10.1371/journal.pone.0208122 (2018).
21 Milane, L. & Amiji, M. Clinical approval of nanotechnology-based SARS-COV-2 mRNA vaccines: impact on translational nanomedicine. Drug Deliv Transl Res, doi:10.1007/s13346-021-00911-y (2021).
22 Corbett, K. S. et al. SARS-COV-2 mRNA Vaccine Development Enabled by Prototype Pathogen Preparedness. bioRxiv, doi:10.1101/2020.06.11.145920 (2020).
23 Corbett, K. S. et al. Evaluation of the mRNA-1273 Vaccine against SARS-COV-2 in Nonhuman Primates. N Engl J Med, doi:10.1056/NEJMoa2024671 (2020).
24 Dong, Y. et al. A systematic review of SARS-COV-2 vaccine candidates. Signal Transduction and Targeted Therapy 5, 237, doi:10.1038/s41392-020-00352-y (2020).
25 Fuller, D. H. & Berglund, P. Amplifying RNA Vaccine Development. N Engl J Med 382, 2469-2471, doi:10.1056/NEJMcibr2009737 (2020).
26 Han, H. J., Nwagwu, C., Anyim, O., Ekweremadu, C. & Kim, S. COVID-19 and cancer: From basic mechanisms to vaccine development using nanotechnology. International immunopharmacology 90, 107247, doi:10.1016/j.intimp.2020.107247 (2021).
27 Huang, L. et al. SARS-COV-2 vaccine research and development: Conventional vaccines and biomimetic nanotechnology strategies. Asian Journal of Pharmaceutical Sciences, doi.org/10.1016/j.ajps.2020.08.001 (2020).
28 Keech, C. et al. Phase 1-2 Trial of a SARS-COV-2 Recombinant Spike Protein Nanoparticle Vaccine. New England Journal of Medicine 383, 2320-2332, doi:10.1056/NEJMoa2026920 (2020).
29 Kim, J. H., Marks, F. & Clemens, J. D. Looking beyond COVID-19 vaccine phase 3 trials. Nat Med 27, 205-211, doi:10.1038/s41591-021-01230-y (2021).
30 Lundstrom, K. The Current Status of COVID-19 Vaccines. Frontiers in Genome Editing 2, doi:10.3389/fgeed.2020.579297 (2020).
31 Wolfram, J. & Ferrari, M. Clinical Cancer Nanomedicine. Nano today 25, 85-98, doi:10.1016/j.nantod.2019.02.005 (2019).
32 Milane, L., Dolare, S., Jahan, T. & Amiji, M. Mitochondrial nanomedicine: Subcellular organelle-specific delivery of molecular medicines. Nanomedicine 37, 102422, doi:10.1016/j.nano.2021.102422 (2021).
33 Valente, A. J., Maddalena, L. A., Robb, E. L., Moradi, F. & Stuart, J. A. A simple ImageJ macro tool for analyzing mitochondrial network morphology in mammalian cell culture. Acta Histochem 119, 315-326, doi:10.1016/j.acthis.2017.03.001 (2017).
34 Koshiba, T. et al. Structural basis of mitochondrial tethering by mitofusin complexes. Science 305, 858-862, doi:10.1126/science. 1099793 (2004).

### REFERENCE LIST 2

1 Gadhave, D. G. et al. Neurodegenerative disorders: Mechanisms of degeneration and therapeutic approaches with their clinical relevance. Ageing Research Reviews 99, 102357, doi.org/10.1016/j.arr.2024.102357 (2024).
2 Bustamante-Barrientos, F. A. et al. Mitochondrial dysfunction in neurodegenerative disorders: Potential therapeutic application of mitochondrial transfer to central nervous system-residing cells. Journal of Translational Medicine 21, 613, doi:10.1186/s12967-023-04493-w (2023).
3 Lei, J. & Gillespie, K. Projected Global Burden of Brain Disorders Through 2050 (P7-15.001). Neurology 102, 3234, doi:10.1212/WNL.0000000000205009 (2024).
4 Hou, Y. et al. Ageing as a risk factor for neurodegenerative disease. Nature Reviews Neurology 15, 565-581, doi:10.1038/s41582-019-0244-7 (2019).
5 Azam, S., Haque, M. E., Balakrishnan, R., Kim, I.-S. & Choi, D.-K. The Ageing Brain: Molecular and Cellular Basis of Neurodegeneration. Front Cell Dev Biol 9, doi:10.3389/fcell.2021.683459 (2021).
6 ADI. Dementia Statistics, www.alzint.org/about/dementia-facts-figures/dementia-statistics/(2025).
7 Understanding Parkinson's, www.parkinson.org/understanding-parkinsons/statistics #:˜:text=Parkinson's%20is%20the%20second%2Dmost,worldwide% 20 are%20living%20with%20PD. (2025).
8 López-Otín, C., Blasco, M. A., Partridge, L., Serrano, M. & Kroemer, G. Hallmarks of aging: An expanding universe. Cell 186, 243-278, doi:10.1016/j.cell.2022.11.001 (2023).
9 Fang, E. F. et al. Mitophagy inhibits amyloid-β and tau pathology and reverses cognitive deficits in models of Alzheimer's disease. Nature Neuroscience 22, 401-412, doi:10.1038/s41593-018-0332-9 (2019).
10 Klemmensen, M. M., Borrowman, S. H., Pearce, C., Pyles, B. & Chandra, B. Mitochondrial dysfunction in neurodegenerative disorders. Neurotherapeutics 21, e00292, doi: doi.org/10.1016/j.neurot.2023.10.002 (2024).
11 Area-Gomez, E., Guardia-Laguarta, C., Schon, E. A. & Przedborski, S. Mitochondria, OxPhos, and neurodegeneration: cells are not just running out of gas. J Clin Invest 129, 34-45, doi:10.1172/jci120848 (2019).
12 Ali, M. D. Z. & Dholaniya, P. S. Oxidative phosphorylation mediated pathogenesis of Parkinson's disease and its implication via Akt signaling. Neurochemistry International 157, 105344, doi: doi.org/10.1016/j.neuint.2022.105344 (2022).
13 Stewart, J. B. & Chinnery, P. F. The dynamics of mitochondrial DNA heteroplasmy: implications for human health and disease. Nature Reviews Genetics 16, 530-542, doi:10.1038/nrg3966 (2015).
14 Misgeld, T. & Schwarz, T. L. Mitostasis in Neurons: Maintaining Mitochondria in an Extended Cellular Architecture. Neuron 96, 651-666, doi:10.1016/j.neuron.2017.09.055 (2017).
15 Grandemange, S., Herzig, S. & Martinou, J.-C. Mitochondrial dynamics and cancer. Seminars in Cancer Biology 19, 50-56, doi: doi.org/10.1016/j.semcancer.2008.12.001 (2009).
16 Maycotte, P. et al. Mitochondrial dynamics and cancer. Tumor Biology 39, 1010428317698391, doi:10.1177/1010428317698391 (2017).
17 Fivenson, E. M. et al. Mitophagy in neurodegeneration and aging. Neurochemistry International 109, 202-209, doi: doi.org/10.1016/j.neuint.2017.02.007 (2017).
18 Wang, S. et al. The mitophagy pathway and its implications in human diseases. Signal Transduction and Targeted Therapy 8, 304, doi:10.1038/s41392-023-01503-7 (2023).
19 Filadi, R., Pendin, D. & Pizzo, P. Mitofusin 2: from functions to disease. Cell Death & Disease 9, 330, doi:10.1038/s41419-017-0023-6 (2018).
20 Scorrano, L. Keeping mitochondria in shape: a matter of life and death. European Journal of Clinical Investigation 43, 886-893, doi:10.1111/eci. 12135 (2013).
21 Franco, A. et al. Correcting mitochondrial fusion by manipulating mitofusin conformations. Nature 540, 74-79, doi:10.1038/nature20156 (2016).
22 Landes, T. & Martinou, J.-C. Mitochondrial outer membrane permeabilization during apoptosis: The role of mitochondrial fission. Biochimica et Biophysica Acta (BBA)—Molecular Cell Research 1813, 540-545, doi: doi.org/10.1016/j.bbamcr.2011.01.021 (2011).
23 Renault, Thibaud T. et al. Mitochondrial Shape Governs BAX-Induced Membrane Permeabilization and Apoptosis. Molecular Cell 57, 69-82, doi:10.1016/j.molcel.2014.10.028 (2015).
24 Chen, H., McCaffery, J. M. & Chan, D. C. Mitochondrial fusion protects against neurodegeneration in the cerebellum. Cell 130, 548-562, doi:10.1016/j.cell.2007.06.026 (2007).
25 Yuan, H. et al. Mitochondrial fission is an upstream and required event for bax foci formation in response to nitric oxide in cortical neurons. Cell death and differentiation 14, 462-471, doi:10.1038/sj.cdd.4402046 (2007).
26 Karbowski, M. et al. Spatial and temporal association of Bax with mitochondrial fission sites, Drp1, and Mfn2 during apoptosis. The Journal of cell biology 159, 931-938, doi:10.1083/jcb.200209124 (2002).
27 Neuspiel, M., Zunino, R., Gangaraju, S., Rippstein, P. & McBride, H. Activated mitofusin 2 signals mitochondrial fusion, interferes with Bax activation, and reduces susceptibility to radical induced depolarization. The Journal of biological chemistry 280, 25060-25070, doi:10.1074/jbc.M501599200 (2005).
28 Frank, S. et al. The role of dynamin-related protein 1, a mediator of mitochondrial fission, in apoptosis. Developmental cell 1, 515-525, doi:10.1016/s1534-5807 (01) 00055-7 (2001).
29 Guo, J. et al. Aging and aging-related diseases: from molecular mechanisms to interventions and treatments. Signal Transduction and Targeted Therapy 7, 391, doi:10.1038/s41392-022-01251-0 (2022).
30 Koszła, O. & Sołek, P. Misfolding and aggregation in neurodegenerative diseases: protein quality control machinery as potential therapeutic clearance pathways. Cell Communication and Signaling 22, 421, doi:10.1186/s12964-024-01791-8 (2024).
31 Wu, J., Wu, J., Chen, T., Cai, J. & Ren, R. Protein aggregation and its affecting mechanisms in neurodegenerative diseases. Neurochemistry International 180, 105880, doi: doi.org/10.1016/j.neuint.2024.105880 (2024).
32 Gandhi, J. et al. Protein misfolding and aggregation in neurodegenerative diseases: a review of pathogeneses, novel detection strategies, and potential therapeutics. Rev Neurosci 30, 339-358, doi:10.1515/revneuro-2016-0035 (2019).
33 Bravo-Sagua, R. et al. Cell death and survival through the endoplasmic reticulum-mitochondrial axis. Curr Mol Med 13, 317-329, doi:10.2174/156652413804810781 (2013).
34 de Brito, O. M. & Scorrano, L. Mitofusin 2 tethers endoplasmic reticulum to mitochondria. Nature 456, 605, doi:10.1038/nature07534 www.nature.com/articles/nature07534 #supplementary-information (2008).
35 Martinvalet, D. The role of the mitochondria and the endoplasmic reticulum contact sites in the development of the immune responses. Cell Death & Disease 9, 336, doi:10.1038/s41419-017-0237-7 (2018).
36 Naon, D. et al. Critical reappraisal confirms that Mitofusin 2 is an endoplasmic reticulum-mitochondria tether. Proceedings of the National Academy of Sciences 113, 11249 (2016).
37 Williamson, C. D., Wong, D. S., Bozidis, P., Zhang, A. & Colberg-Poley, A. M. Isolation of Endoplasmic Reticulum, Mitochondria, and Mitochondria-Associated Membrane and Detergent Resistant Membrane Fractions from Transfected Cells and from Human Cytomegalovirus-Infected Primary Fibroblasts. Current protocols in cell biology/editorial board, Juan S. Bonifacino . . . [et al.] 68, 3.27.21-23.27.33, doi:10.1002/0471143030.cb0327s68 (2015).
38 Bravo, R. et al. Endoplasmic Reticulum and the Unfolded Protein Response: Dynamics and Metabolic Integration. International review of cell and molecular biology 301, 215-290, doi:10.1016/B978-0-12-407704-1.00005-1 (2013).
39 McGrath, E. P. et al. The Unfolded Protein Response in Breast Cancer. Cancers 10, 344 (2018).
40 Oslowski, C. M. & Urano, F. Measuring ER stress and the unfolded protein response using mammalian tissue culture system. Methods in enzymology 490, 71-92, doi:10.1016/B978-0-12-385114-7.00004-0 (2011).
41 Peñaranda Fajardo, N. M., Meijer, C. & Kruyt, F. A. E. The endoplasmic reticulum stress/unfolded protein response in gliomagenesis, tumor progression and as a therapeutic target in glioblastoma. Biochemical Pharmacology 118, 1-8, doi: doi.org/10.1016/j.bcp.2016.04.008 (2016).
42 Milisav, I., Šuput, D. & Ribarič, S. Unfolded Protein Response and Macroautophagy in Alzheimer's, Parkinson's and Prion Diseases. Molecules 20, doi:10.3390/molecules201219865 (2015).
43 Bravo, R. et al. Increased ER-mitochondrial coupling promotes mitochondrial respiration and bioenergetics during early phases of ER stress. Journal of Cell Science 124, 2143-2152, doi:10.1242/jcs.080762 (2011).
44 Ajoolabady, A., Lindholm, D., Ren, J. & Pratico, D. ER stress and UPR in Alzheimer's disease: mechanisms, pathogenesis, treatments. Cell Death & Disease 13, 706, doi:10.1038/s41419-022-05153-5 (2022).
45 Nagar, P. et al. Endoplasmic reticulum stress in Alzheimer's disease: Molecular mechanisms and therapeutic prospects. Life Sciences 330, 121983, doi: doi.org/10.1016/j.lfs.2023.121983 (2023).
46 Valente, A. J., Maddalena, L. A., Robb, E. L., Moradi, F. & Stuart, J. A. A simple ImageJ macro tool for analyzing mitochondrial network morphology in mammalian cell culture. Acta Histochem 119, 315-326, doi:10.1016/j.acthis.2017.03.001 (2017).
47 Milane, L. S., Dolare, S., Ren, G. & Amiji, M. Combination Organelle Mitochondrial Endoplasmic Reticulum Therapy (COMET) for Multidrug Resistant Breast Cancer. J Control Release 363, 435-451, doi:10.1016/j.jconrel.2023.09.023 (2023).
48 Pawar, G. N., Parayath, N. N., Nocera, A. L., Bleier, B. S. & Amiji, M. M. Direct CNS delivery of proteins using thermosensitive liposome-in-gel carrier by heterotopic mucosal engrafting. PLOS ONE 13, e0208122, doi:10.1371/journal.pone.0208122 (2018).

1. A composition for enhancing a mitochondrial network in a cell, the composition comprising:
a plurality of nanoparticles each comprising a mitochondrial network enhancing agent;
wherein the mitochondrial network enhancing agent enhances the mitochondrial network of the cell when the nanoparticles are taken up by the cell.

2. The composition of claim 1, wherein the mitochondrial network enhancing agent binds to a mitofusin protein on mitochondria of the cell.

3. The composition of claim 2, wherein the mitofusin protein is mitofusin 1 (MFN1) or mitofusin 2 (MFN2).

4. The composition of claim 3, wherein the mitofusin protein is MFN2.

5. The composition of claim 3, wherein the mitochondrial network enhancing agent binds to MFN1 or MFN2 and thereby promotes fusion of mitochondria of the cell with one another and/or with endoplasmic reticulum of the cell.

6. The composition of claim 5, wherein the mitochondrial network enhancing agent is a peptide fragment of MFN1 or MFN2.

7. The composition of claim 6, wherein the mitochondrial network enhancing agent is a peptide fragment of MFN2 and comprises the amino acid sequence DIAEAVRLIMDSLHMAAR (SEQ ID NO:1), or a fragment or variant thereof having at least 70%, at least 80%, at least 90%, at least 95%, at least 98%, or at least 99% identity to SEQ ID NO:1.

8. The composition of claim 7, wherein the mitochondrial network enhancing agent is a variant of SEQ ID NO: 1 comprising from 1 to 10, from 1 to 5, or from 1 to 3 conservative amino acid substitutions compared to SEQ ID NO:1.

9. The composition of claim 1, wherein the nanoparticles comprise lipid nanoparticles or biodegradable polymeric nanoparticles, and wherein the lipid nanoparticles or polymeric nanoparticles encapsulate the mitochondrial network enhancing agent.

10. The composition of claim 9, wherein the nanoparticles comprise lipid nanoparticles, and wherein the lipid nanoparticles are liposomes.

11. The composition of claim 10, wherein the liposomes are cationic liposomes.

12. The composition of claim 11, wherein the cationic liposomes comprise 1,2-dioleoyl-3-trimethylammonium-propane (DOTAP) as the cationic lipid and further comprise one or more components selected from the group consisting of a neutral phospholipid, cholesterol, and a hydrophilic polymer such as polyethylene glycol (PEG).

13. The composition of claim 11, wherein the cationic liposomes have a zeta potential greater than about +30 mV or less than about −30 mV.

14. The composition of claim 9, wherein the nanoparticles are biodegradable polymeric nanoparticles.

15. The composition of claim 14, wherein the biodegradable polymeric nanoparticles comprise one or more biodegradable polymeric polymers selected from the group consisting of gelatin, chitosan, cellulose, cellulose derivatives, poly(ε-caprolactone) (PCL), polyethylene glycol (PEG), poly(lactic-co-glycolic acid) (PLGA), polylactic acid (PLA), polyglycolide, PEG-PLA diblock copolymer, PEG-PLGA diblock copolymer, PEG-PCL diblock copolymer, PCL-b-PEG-b-PCL co-polymer, polypropylene glycol (PPG), polyacrylic acid, polyacrylamide, poly(N-isopropylacrylamide), polyethylene oxide-polypropylene oxide-polyethylene oxide (PEO-PPO-PEO) triblock copolymer, hyaluronic acid, polyethylene oxide, polypropylene oxide, alginic acid, poly(betaaminoester) (PBAE), poly(glycolic acid) (PGA), and alginate.

16. A method of enhancing a mitochondrial network in a cell of a mammalian subject, the method comprising:
(a) providing the composition of any of the claim 1; and
(b) administering the composition to the mammalian subject, whereby the mitochondrial network is enhanced in the cell of the mammalian subject.

17. The method of claim 16, wherein said enhancing the mitochondrial network in the cell of the mammalian subject comprises enhancing fusion of mitochondria with other mitochondria and/or with endoplasmic reticulum in the cell, and/or increasing a size or density of the mitochondrial network in the cell.

18. The method of claim 16, wherein said enhancing the mitochondrial network in the cell of the mammalian subject results in one or more of increased energy production, reduced reactive oxygen species (ROS) production, reduced nuclear DNA damage, reduced mitochondrial DNA damage, increased cell survival, increased protein folding efficiency, reduced protein misfolding, reduced accumulation of misfolded proteins, increased autophagy, increased mitophagy, and reduced apoptosis.

19. The method of claim 16, wherein said enhancing the mitochondrial network in the cell of the mammalian subject comprises reducing fission of mitochondria in cells of the mammalian subject.

20. A method of enhancing a mitochondrial network in a cell, the method comprising:
(a) providing the composition of claim 1 and a culture comprising the cell; and
(b) adding the composition to a medium of the culture, whereby nanoparticles of the composition are taken up by the cell and the mitochondrial network in the cell is enhanced.


SEQUENCE LISTING (1 sequence)

SEQ ID NO: 1 - protein, 18 aa, Homo sapiens
DIAEAVRLIMDSLHMAAR